(19) United States
(12) Patent Application Publication
(10) Pub. No.: US 2024/0041828 A1
(43) Pub. Date: Feb. 8, 2024

(54) Combination Therapeutics Comprising Nrf2 Agonists and Antivirals for Treating Viral Infections

(71) Applicant: The Regents of the University of California, Oakland, CA (US)

(72) Inventor: Theodoros Kelesidis, Dallas, TX (US)

(21) Appl. No.: 18/353,069

(22) Filed: Jul. 15, 2023

Related U.S. Application Data
(60) Provisional application No. 63/393,802, filed on Jul. 29, 2022.

(51) Int. Cl. A61K 31/403; A61K 31/426; A61K 31/497; A61K 31/215; A61K 31/66; A61P 31/14

(52) U.S. Cl. CPC A61K 31/403; A61K 31/426; A61K 31/497; A61K 31/215; A61K 31/66; A61P 31/14

(57) ABSTRACT

Disclosed herein are compositions, kits, and methods employing combinations of (a) one or more Nrf2 agonists; and (b) one or more 3CLpro inhibitors, one or more protease inhibitors, and/or one or more N⁴-hydroxycytidine nucleoside to treat, inhibit, and/or reduce infections and symptoms caused by infection by a virus such as viruses belonging to the Coronaviridae family (e.g., coronaviruses), Orthomyxoviridae family, the Picornaviridae family, and the Pneumoviridae family of viruses.

### CROSS-REFERENCE TO RELATED APPLICATIONS

This application claims the benefit of U.S. Patent Application No. 63/393,802, filed Jul. 29, 2022, which is herein incorporated by reference in its entirety.

### BACKGROUND OF THE INVENTION

### 1. Field of the Invention

The field of the invention generally relates to compositions and methods for inhibiting and/or treating infections by viruses belonging to the Coronaviridae family (e.g., coronaviruses), Orthomyxoviridae family, the Picornaviridae family, and the Pneumoviridae family of viruses.

### 2. Description of the Related Art

The COVID-19 pandemic is evidence that the development of vaccines and antivirals is challenging because coronaviruses such as SARS-CoV-2 have high mutation rates. The pandemic also brought to light that many vaccines such as the COVID vaccines have suboptimal efficacy, especially in immunocompromised patients and against divergent strains. Additionally, current antivirals against SARS-CoV-2 have major limitations and cannot be used long term in high-risk groups such as immunocompromised patients. Thus, a need exists for a therapeutic that may be administered for long periods of time, e.g., chronic administration, as a pre- or post-exposure prophylactic and/or as an antiviral post-infection that does not depend on learned immunity.

### SUMMARY OF THE INVENTION

In some embodiments, the present invention is directed to a composition, which comprises, consists essentially of, or consists of (a) one or more Nrf2 agonists; and (b) one or more 3CLpro inhibitors, one or more protease inhibitors, and/or one or more N⁴-hydroxycytidine nucleosides. In some embodiments, the one or more Nrf2 agonists is (a) a TPP Compound, a mitochondrial targeted antioxidant, or dimethyl fumarate (DMF); (b) mitoquinone mesylate and/or mitoquinol mesylate; (c) DMF; or (d) (i) DMF, and (ii) mitoquinone mesylate and/or mitoquinol mesylate. In some embodiments, the 3CLpro inhibitor is a nirmatrelvir compound. In some embodiments, the protease inhibitor is a ritonavir compound. In some embodiments, the N⁴-hydroxycytidine nucleoside is molnupiravir. In some embodiments, the 3CLpro inhibitor is a nirmatrelvir compound. In some embodiments, the protease inhibitor is a ritonavir compound. In some embodiments, the N⁴-hydroxycytidine nucleoside is molnupiravir. In some embodiments, the composition comprises (a) a TPP Compound; (b) DMF, and (c)(i) nirmatrelvir and ritonavir; or (ii) molnupiravir.

In some embodiments, the present invention is directed to a method of treating, inhibiting, and/or reducing an infection by a virus or a symptom caused by the infection in a subject, which comprises, consists essentially of, or consists of administering (a) one or more Nrf2 agonists; and (b) one or more 3CLpro inhibitors, one or more protease inhibitors, and/or one or more N⁴-hydroxycytidine nucleosides to the subject. In some embodiments, the one or more Nrf2 agonists is (a) a TPP Compound, a mitochondrial targeted antioxidant, or dimethyl fumarate (DMF); (b) mitoquinone mesylate and/or mitoquinol mesylate; (c) DMF; or (d) (i) DMF, and (ii) mitoquinone mesylate and/or mitoquinol mesylate. In some embodiments, the 3CLpro inhibitor is a nirmatrelvir compound. In some embodiments, the protease inhibitor is a ritonavir compound. In some embodiments, the N⁴-hydroxycytidine nucleoside is molnupiravir. In some embodiments, the 3CLpro inhibitor is a nirmatrelvir compound. In some embodiments, the protease inhibitor is a ritonavir compound. In some embodiments, the N⁴-hydroxycytidine nucleoside is molnupiravir. In some embodiments, the method comprises administering to the subject (a) a TPP Compound; (b) DMF; and (c)(i) nirmatrelvir and ritonavir, or (ii) molnupiravir. In some embodiments, the virus is a coronavirus. In some embodiments, the virus is SARS-CoV-2. In some embodiments, the symptom caused by the infection is cytotoxic injury, an aberrant host inflammatory response, and/or lung injury.

In some embodiments, the present invention is directed to a kit which comprises, consists essentially of, or consists of (a) one or more Nrf2 agonists; and (b) one or more 3CLpro inhibitors, one or more protease inhibitors, and/or one or more N⁴-hydroxycytidine nucleosides packaged together. In some embodiments, the one or more Nrf2 agonists is (a) a TPP Compound, a mitochondrial targeted antioxidant, or dimethyl fumarate (DMF); (b) mitoquinone mesylate and/or mitoquinol mesylate; (c) DMF; or (d) (i) DMF, and (ii) mitoquinone mesylate and/or mitoquinol mesylate. In some embodiments, the 3CLpro inhibitor is a nirmatrelvir compound. In some embodiments, the protease inhibitor is a ritonavir compound. In some embodiments, the N⁴-hydroxycytidine nucleoside is molnupiravir. In some embodiments, the 3CLpro inhibitor is a nirmatrelvir compound. In some embodiments, the protease inhibitor is a ritonavir compound. In some embodiments, the N⁴-hydroxycytidine nucleoside is molnupiravir. In some embodiments, the kit comprises, packaged together, (a) a TPP Compound; (b) DMF; and (c)(i) nirmatrelvir and ritonavir, or (ii) molnupiravir.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

This invention is further understood by reference to the drawings wherein:

FIG. 1. Mitoquinone mesylate (Mito-MES) has strong anti-SARS-CoV-2 activity in single layer human airway cells that is similar to Paxlovid™ (nirmatrelvir/ritonavir). High ACE2 (hACE2) A549 cells were infected with B.1.617.2 (Delta) SARS-CoV-2 variant at an MOI of 0.5 and were treated with drugs for 2 hours before infection and throughout the experiment. Viral replication at 24 hours post infection (hpi) by measurement of intracellular SARS-CoV-2 nucleocapsid protein (NP) by ELISA (hACE2-A549 cells). Cell cytotoxicity was assessed in supernatant of cells by the LDH assay (24 h). Half maximal inhibitory concentration (IC50), 50% cytotoxic concentration (CC50) values for drugs are indicated. In all panels, data are means ±standard error of the mean (SEM) of at least three independent experiments with at least 3 replicates in each experiment.

FIG. 2. Mitoquinone mesylate (Mito-MES) has stronger anti-SARS-CoV-2 activity in multilayer human airway cell culture than Paxlovid™ (nirmatrelvir/ritonavir). Human bronchial airway epithelial (HBEC) cells culture in air liquid interface (ALI) cultures were infected with SARS-coV-2 Omicron variant at an MOI of 0.1 and were treated with drugs for 2 hours before infection and throughout the experiment. Viral replication at 24 hours post infection (hpi) by measurement of intracellular SARS-CoV-2 nucleocapsid protein (NP) by ELISA. Cell cytotoxicity was assessed in supernatant of cells by the LDH assay (24 h). Half maximal inhibitory concentration (IC50), 50% cytotoxic concentration (CC50) values for drugs are indicated. In all panels, data are means±standard error of the mean (SEM) of at least three independent experiments with at least 3 replicates in each experiment.

FIG. 3 and FIG. 4. Mitoquinone mesylate (Mito-MES, “MTQ”) has additive anti-SARS-CoV-2 activity with Paxlovid™ (nirmatrelvir/ritonavir) in single layer human airway cells. High ACE2 (hACE2) A549 cells were infected with B.1.617.2 (Delta) SARS-CoV-2 variant at an MOI of 0.5 and were treated with drugs for 2 hours before infection and throughout the experiment. Viral replication at 24 hours post infection (hpi) by measurement of intracellular SARS-CoV-2 nucleocapsid protein (NP) by ELISA (hACE2-A549 cells). % inhibition compared to DMSO vehicle control for drugs are indicated. Data are means±standard error of the mean (SEM) of at least three independent experiments with at least 3 replicates in each experiment.

FIG. 5. Dimethyl fumarate (DMF) has anti-SARS-CoV-2 activity in single layer human airway cells that is lower than Paxlovid™ (nirmatrelvir/ritonavir). High ACE2 (hACE2) A549 cells were infected with B.1.617.2 (Delta) SARS-CoV-2 variant at an MOI of 0.5 and were treated with drugs for 2 hours before infection and throughout the experiment. Viral replication at 24 hours post infection (hpi) by measurement of intracellular SARS-CoV-2 nucleocapsid protein (NP) by ELISA (hACE2-A549 cells). Cell cytotoxicity was assessed in supernatant of cells by the LDH assay (24 h). Half maximal inhibitory concentration (IC50), 50% cytotoxic concentration (CC50) values for drugs are indicated. In all panels, data are means±standard error of the mean (SEM) of at least three independent experiments with at least 3 replicates in each experiment.

FIG. 6. Dimethyl-fumarate (DMF) has similar to Paxlovid™ (nirmatrelvir/ritonavir) anti-SARS-CoV-2 activity in multilayer human airway cell culture. Human bronchial airway epithelial (HBEC) cells culture in air liquid interface (ALI) cultures were infected with SARS-coV-2 Omicron variant at an MOI of 0.1 and were treated with drugs for 2 hours before infection and throughout the experiment. Viral replication at 24 hours post infection (hpi) by measurement of intracellular SARS-CoV-2 nucleocapsid protein (NP) by ELISA. Cell cytotoxicity was assessed in supernatant of cells by the LDH assay (24 h). Half maximal inhibitory concentration (IC50), 50% cytotoxic concentration (CC50) values for drugs are indicated. In all panels, data are means±standard error of the mean (SEM) of at least three independent experiments with at least 3 replicates in each experiment.

FIG. 7 and FIG. 8. Dimethyl fumarate (DMF) has additive anti-SARS-CoV-2 activity with Paxlovid™ (nirmatrelvir/ritonavir) in single layer human airway cells. High ACE2 (hACE2) A549 cells were infected with B.1.617.2 (Delta) SARS-CoV-2 variant at an MOI of 0.5 and were treated with drugs for 2 hours before infection and throughout the experiment. Viral replication at 24 hours post infection (hpi) by measurement of intracellular SARS-CoV-2 nucleocapsid protein (NP) by ELISA (hACE2-A549 cells). % inhibition compared to DMSO vehicle control for drugs are indicated. Data are means±standard error of the mean (SEM) of at least three independent experiments with at least 3 replicates in each experiment.

FIG. 9. Mitoquinone mesylate (Mito-MES) has strong anti-SARS-CoV-2 activity in single layer human airway cells that is higher than molnupiravir. High ACE2 (hACE2) A549 cells were infected with B.1.617.2 (Delta) SARS-CoV-2 variant at an MOI of 0.5 and were treated with drugs for 2 hours before infection and throughout the experiment. Viral replication at 24 hours post infection (hpi) by measurement of intracellular SARS-CoV-2 nucleocapsid protein (NP) by ELISA (hACE2-A549 cells). Cell cytotoxicity was assessed in supernatant of cells by the LDH assay (24 h). Half maximal inhibitory concentration (IC50), 50% cytotoxic concentration (CC50) values for drugs are indicated. In all panels, data are means±standard error of the mean (SEM) of at least three independent experiments with at least 3 replicates in each experiment.

FIG. 10. Mitoquinone mesylate (Mito-MES) has stronger anti-SARS-CoV-2 activity in multilayer human airway cell culture than molnupiravir. Human bronchial airway epithelial (HBEC) cells culture in air liquid interface (ALI) cultures were infected with SARS-coV-2 Omicron variant at an MOI of 0.1 and were treated with drugs for 2 hours before infection and throughout the experiment. Viral replication at 24 hours post infection (hpi) by measurement of intracellular SARS-CoV-2 nucleocapsid protein (NP) by ELISA. Cell cytotoxicity was assessed in supernatant of cells by the LDH assay (24 h). Half maximal inhibitory concentration (IC50), 50% cytotoxic concentration (CC50) values for drugs are indicated. In all panels, data are means±standard error of the mean (SEM) of at least three independent experiments with at least 3 replicates in each experiment.

FIG. 11 and FIG. 12. Mitoquinone mesylate (Mito-MES) has additive anti-SARS-CoV-2 activity with molnupiravir in single layer human airway cells. High ACE2 (hACE2) A549 cells were infected with B.1.617.2 (Delta) SARS-CoV-2 variant at an MOI of 0.5 and were treated with drugs for 2 hours before infection and throughout the experiment. Viral replication at 24 hours post infection (hpi) by measurement of intracellular SARS-CoV-2 nucleocapsid protein (NP) by ELISA (hACE2-A549 cells). % inhibition compared to DMSO vehicle control for drugs are indicated. Data are means ±standard error of the mean (SEM) of at least three independent experiments with at least 3 replicates in each experiment.

FIG. 13. Dimethyl fumarate (DMF) has strong anti-SARS-CoV-2 activity in single layer human airway cells that is higher than molnupiravir. High ACE2 (hACE2) A549 cells were infected with B.1.617.2 (Delta) SARS-CoV-2 variant at an MOI of 0.5 and were treated with drugs for 2 hours before infection and throughout the experiment. Viral replication at 24 hours post infection (hpi) by measurement of intracellular SARS-CoV-2 nucleocapsid protein (NP) by ELISA (hACE2-A549 cells). Cell cytotoxicity was assessed in supernatant of cells by the LDH assay (24 h). Half maximal inhibitory concentration (IC50), 50% cytotoxic concentration (CC50) values for drugs are indicated. In all panels, data are means±standard error of the mean (SEM) of at least three independent experiments with at least 3 replicates in each experiment.

FIG. 14. Dimethyl fumarate (DMF) has stronger anti-SARS-CoV-2 activity in multilayer human airway cell culture than molnupiravir. Human bronchial airway epithelial (HBEC) cells culture in air liquid interface (ALI) cultures were infected with SARS-CoV-2 Omicron variant at an MOI of 0.1 and were treated with drugs for 2 hours before infection and throughout the experiment. Viral replication at 24 hours post infection (hpi) by measurement of intracellular SARS-CoV-2 nucleocapsid protein (NP) by ELISA. Cell cytotoxicity was assessed in supernatant of cells by the LDH assay (24 h). Half maximal inhibitory concentration (IC50), 50% cytotoxic concentration (CC50) values for drugs are indicated. In all panels, data are means±SEM of at least three independent experiments with at least 3 replicates in each experiment.

FIG. 15 and FIG. 16. Dimethyl fumarate (DMF) has additive anti-SARS-CoV-2 activity with molnupiravir in single layer human airway cells. High ACE2 (hACE2) A549 cells were infected with B.1.617.2 (Delta) SARS-CoV-2 variant at an MOI of 0.5 and were treated with drugs for 2 hours before infection and throughout the experiment. Viral replication at 24 hours post infection (hpi) by measurement of intracellular SARS-CoV-2 nucleocapsid protein (NP) by ELISA (hACE2-A549 cells). % inhibition compared to DMSO vehicle control for drugs are indicated. Data are means ±standard error of the mean (SEM) of at least three independent experiments with at least 3 replicates in each experiment.

FIG. 17. The combination of two Nrf2 agonists Mito-MES and DMF complement Paxlovid™ (nirmatrelvir/ritonavir) or molnupiravir in vitro to attenuate SARS-CoV-2 replication in lung epithelial cells. High ACE2 (hACE2) A549 cells were infected with B.1.617.2 (Delta) SARS-CoV-2 variant at an MOI of 0.5 and were treated with drugs for 2 hours before infection and throughout the experiment. Viral replication at 24 hours post infection (hpi) by measurement of intracellular SARS-CoV-2 nucleocapsid protein (NP) by ELISA (hACE2-A549 cells). In all panels, data are means ±standard error of the mean (SEM) of at least three independent experiments with at least 3 replicates in each experiment.

FIG. 18. Structures of exemplary TPP Conjugates.

### DETAILED DESCRIPTION OF THE INVENTION

As disclosed in WO 2022015570, which is herein incorporated by reference in its entirety, Nrf2 agonists (e.g., dimethyl fumarate (DMF) and Mito-MES) exert antiviral activity against coronaviruses by agonizing and/or activating the nuclear factor erythroid 2—related factor 2 (Nrf2) signaling pathway. The TPP moiety of Mito-MES was also found to exhibit antiviral activity against coronaviruses by itself. See PCT/US2022/011656, filed Jan. 7, 2022, which is herein incorporated by reference in its entirety. DMF and Mito-MES were also found to exhibit antiviral activity against a plurality of other viruses such as those belonging to the Orthomyxoviridae, Picornaviridae, and Pneumoviridae families of viruses.

Because rebound COVID-19 often occurs after a 5-day course of Paxlovid™ (nirmatrelvir/ritonavir) and some viruses are known to develop a resistance to protease inhibitors, the experiments herein were conducted to determine whether Nrf2 agonists may be co-administered with other antivirals such as Paxlovid™ (nirmatrelvir/ritonavir) and still exhibit antiviral activity without adverse consequences, e.g., cytotoxicity.

### Mito-MES Complements Paxlovid™ In Vitro to Attenuate SARS-CoV-2 Replication in Lung Epithelial Cells In Vitro.

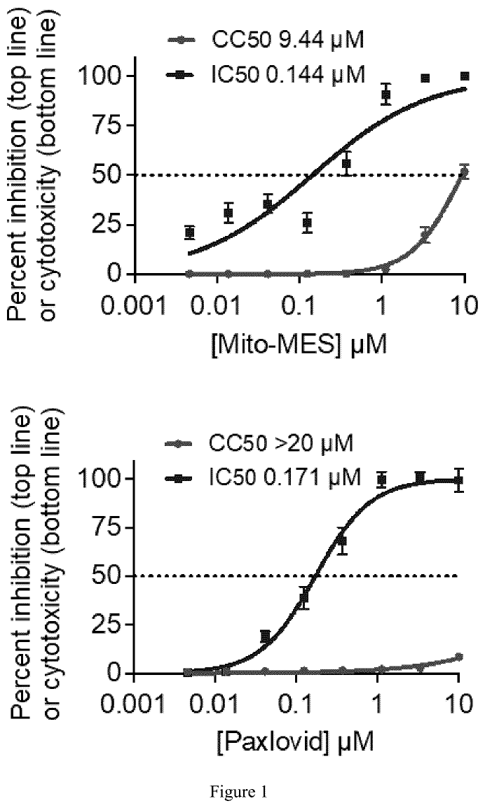

We directly compared the in vitro antiviral activity of Paxlovid™ with Mito-MES in independent lung epithelial cell culture systems. In single layer high ACE2 A549 lung epithelial cells, Mito-MES had similar IC50 compared to Paxlovid™. See FIG. 1.

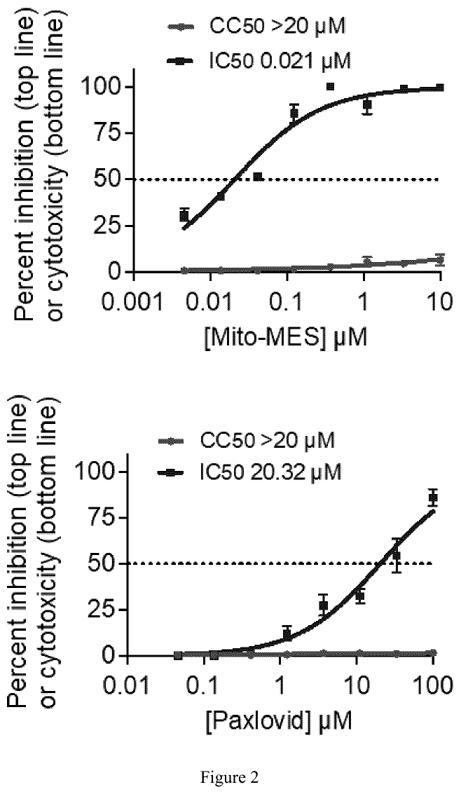

Given emerging data of rebound COVID-19 after 5-day course of Paxlovid™ in patients with COVID-19 and lack of antiviral efficacy data of Paxlovid™ in physiologically relevant air liquid interface (ALI) multilayer airway cell culture system, we hypothesized that Paxlovid™ is less efficacious (maybe due to suboptimal penetration) in three-layer ALI system. We confirmed that indeed the IC50 of Paxlovid™ was about 100-fold less in ALI HAE system compared to single monolayer lung cell system and Mito-MES had about 1000-fold better antiviral effect in ALI HAE system compared to Paxlovid™. See FIG. 2. These data suggest that Paxlovid™ may have suboptimal antiviral efficacy in multilayer tissue interface and combination antiviral drug treatment may be needed to optimize antiviral efficacy in infected lung from SARS-COV-2 in humans.

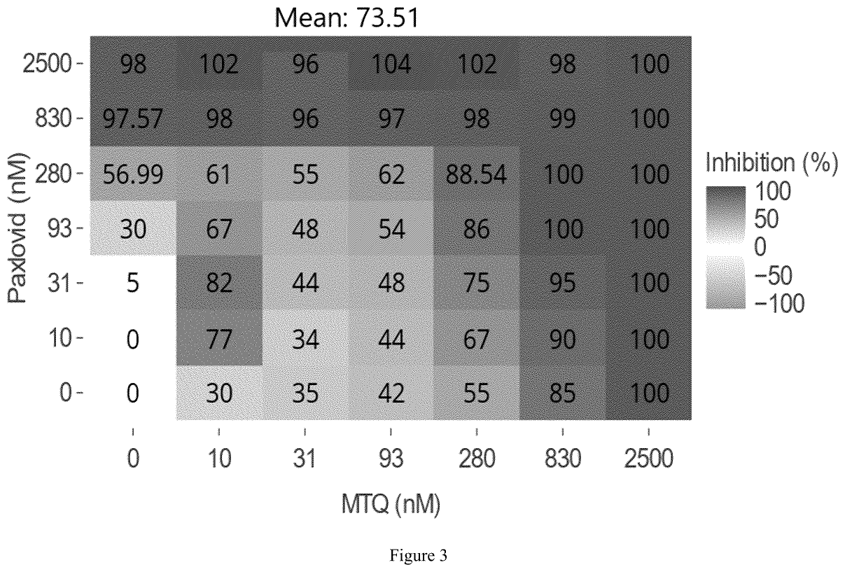

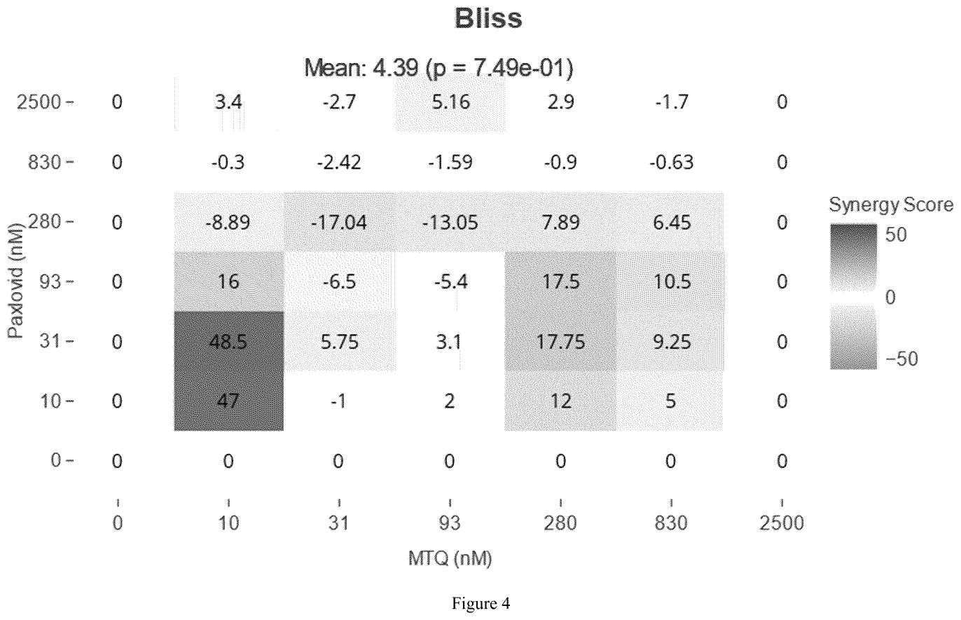

We then performed a dose response matrix (FIG. 3) and synergy distribution analysis (FIG. 4) of Mito-MES (MTQ) and Paxlovid™ in hACE2-A549 cells infected and treated as shown. We used BLISS analysis to determine whether the drugs interact at concentrations 1 nM-2.5 μM. The null hypothesis is that the drugs are additive. A total synergy score from −10 to 10 suggests that the interaction between two drugs is likely to be additive. A mean synergy score 4.4 was obtained suggesting that the Mito-MES and Paxlovid™ have additive antiviral effects.

### DMF Complements Paxlovid™ In Vitro to Attenuate SARS-CoV-2 Replication in Lung Epithelial Cells In Vitro

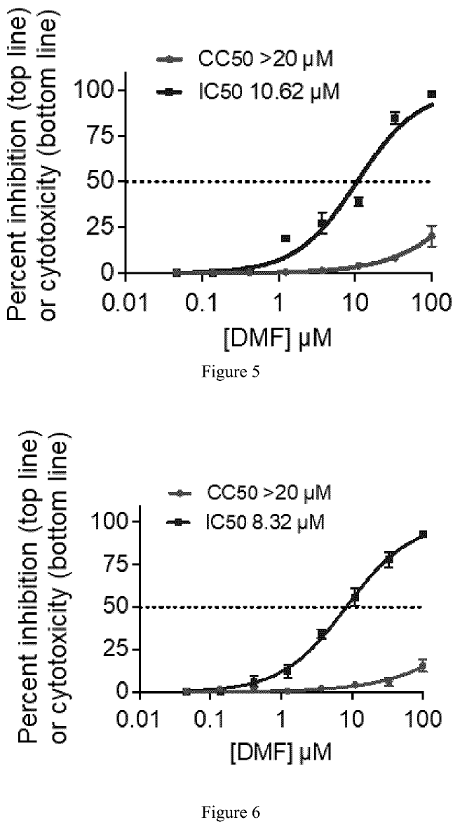

We directly compared the in vitro antiviral activity of Paxlovid™ with DMF in independent lung epithelial cell culture systems. In single layer high ACE2 A549 lung epithelial cells, DMF had higher IC50 compared to Paxlovid™. See FIG. 5. In multilayer cell culture ALI model DMF had IC50 that was similar to Paxlovid™. See FIG. 6.

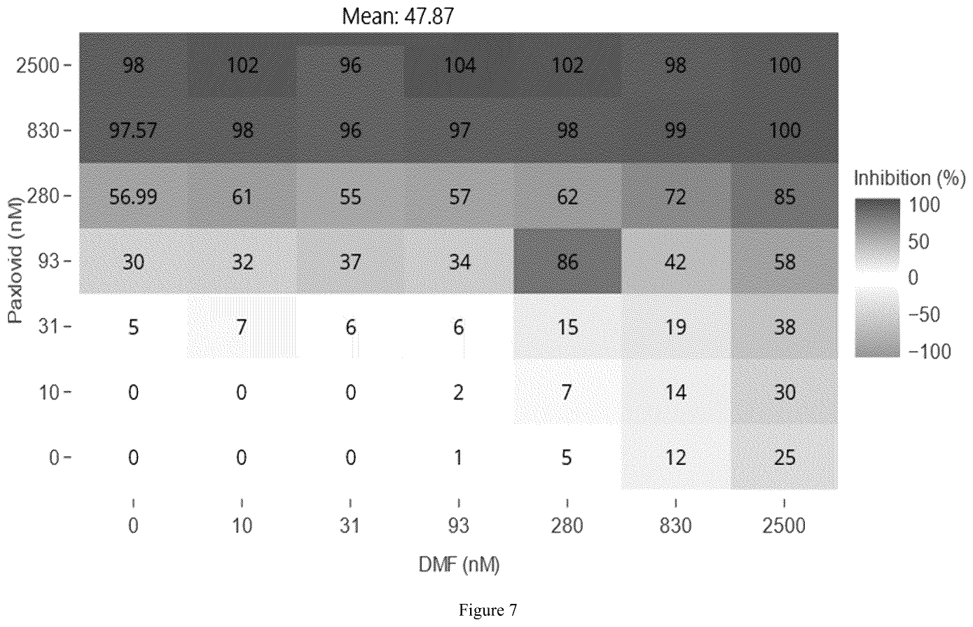

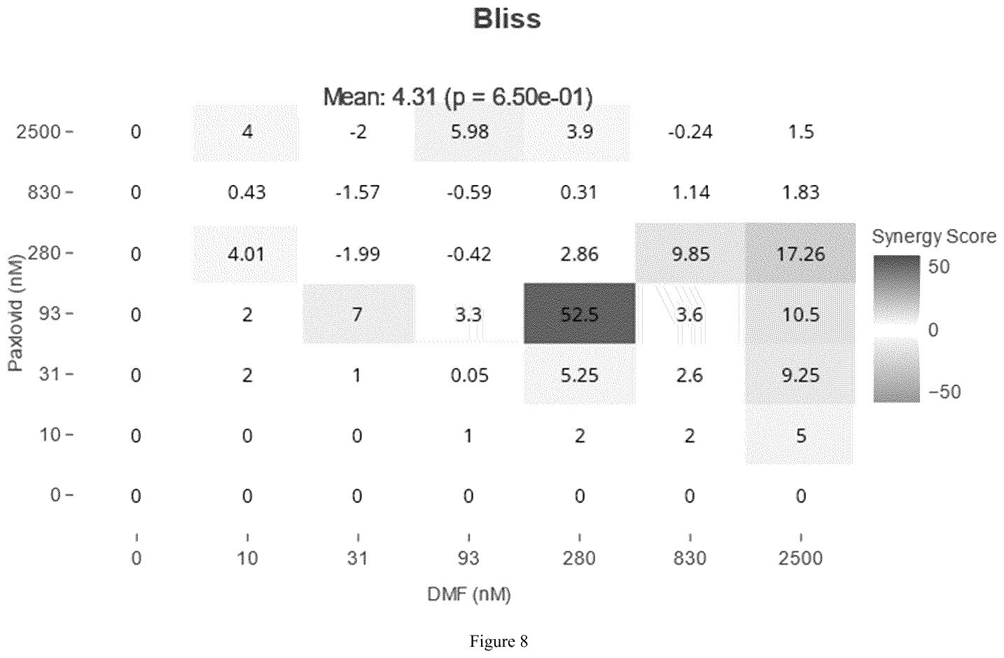

We then performed a dose response matrix (FIG. 7) and synergy distribution analysis (FIG. 8) of DMF and Paxlovid™ in hACE2-A549 cells infected and treated as shown. We used BLISS analysis to determine whether the drugs interact at concentrations 1 nM-2.5 μM. The null hypothesis is that the drugs are additive. A total synergy score from −10 to 10 suggests that the interaction between two drugs is likely to be additive. A mean synergy score 4.3 was obtained suggesting that the DMF and Paxlovid™ have additive antiviral effects.

### Mito-MES Complements Molnupiravir In Vitro to Attenuate SARS-CoV-2 Replication in Lung Epithelial Cells In Vitro.

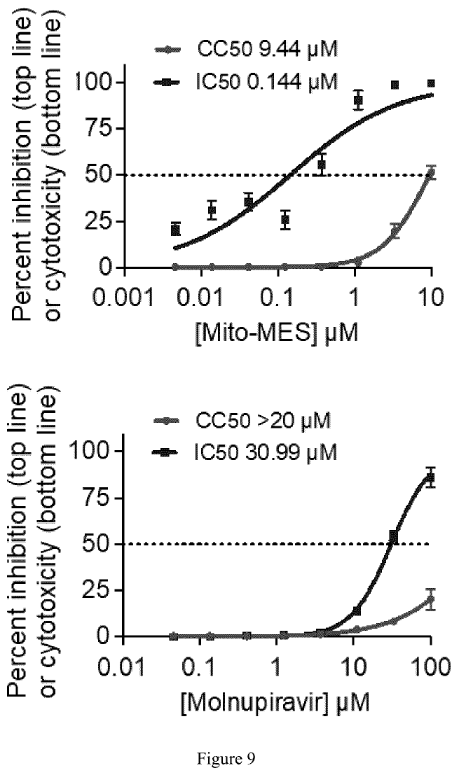

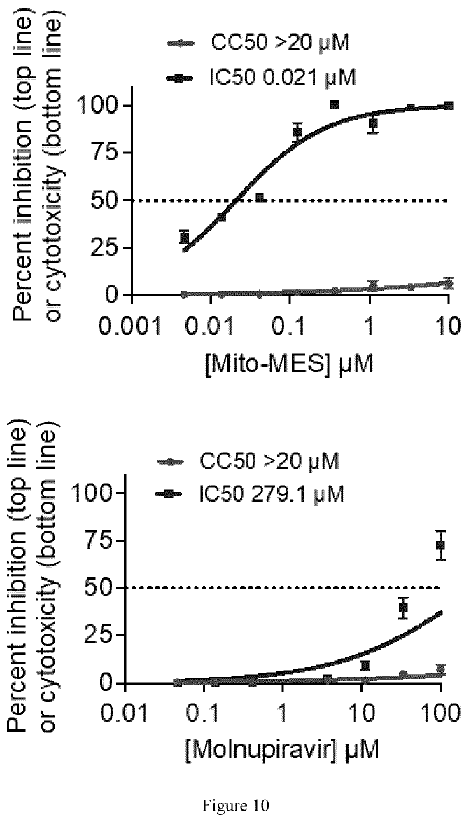

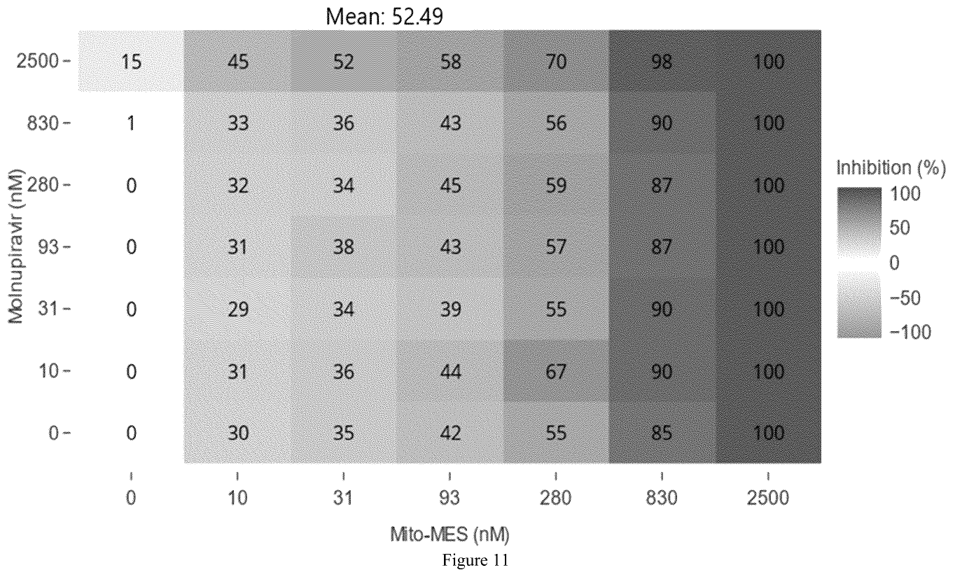

We directly compared the in vitro antiviral activity of molnupiravir with Mito-MES in independent lung epithelial cell culture systems. In single layer high ACE2 A549 lung epithelial cells, Mito-MES had higher IC50 compared to molnupiravir. See FIG. 9 In multilayer, cell culture ALI model Mito-MES had higher than Paxlovid™ IC50. See FIG. 10.

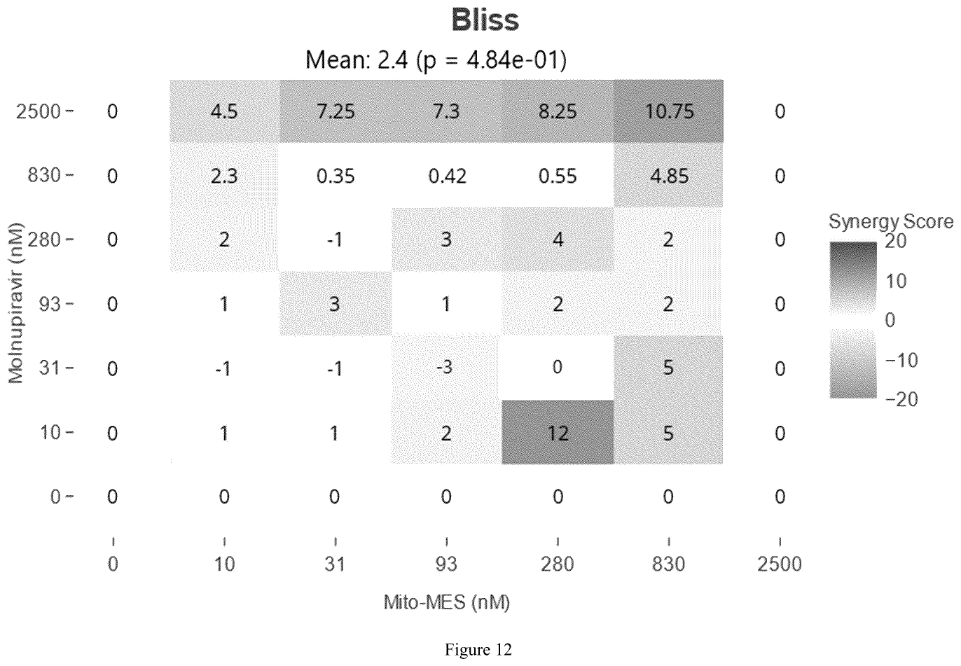

We then performed a dose response matrix (FIG. 11) and synergy distribution analysis (FIG. 12), of Mito-MES and molnupiravir in hACE2-A549 cells infected and treated as shown. We used BLISS analysis to determine whether the drugs interact at concentrations 1 nM to 2.5 μM. The null hypothesis is that the drugs are additive. A total synergy score from −10 to 10 suggests that the interaction between two drugs is likely to be additive. A mean synergy score 2.4 was obtained suggesting that the Mito-MES and molnupiravir have additive antiviral effects.

### DMF Complements Molnupiravir In Vitro to Attenuate SARS-CoV-2 Replication in Lung Epithelial Cells In Vitro.

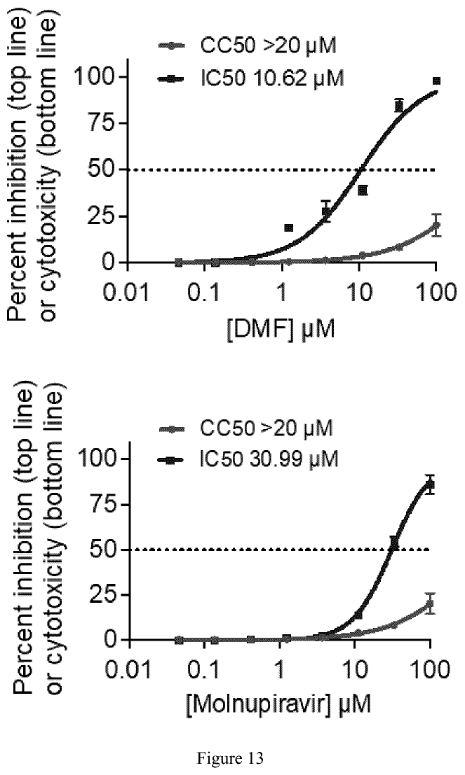

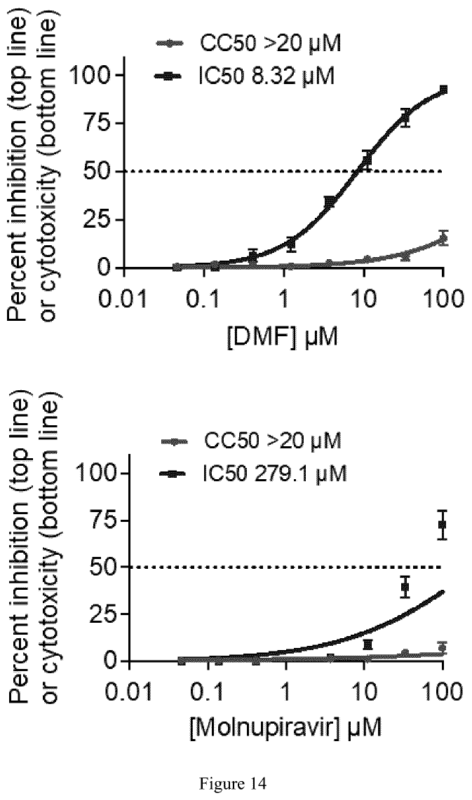

We directly compared the in vitro antiviral activity of molnupiravir with DMF in independent lung epithelial cell culture systems. In single layer high ACE2 A549 lung epithelial cells, DMF had higher IC50 compared to molnupiravir. See FIG. 13. In multilayer cell culture ALI model DMF had higher than molnupiravir IC50. See FIG. 14.

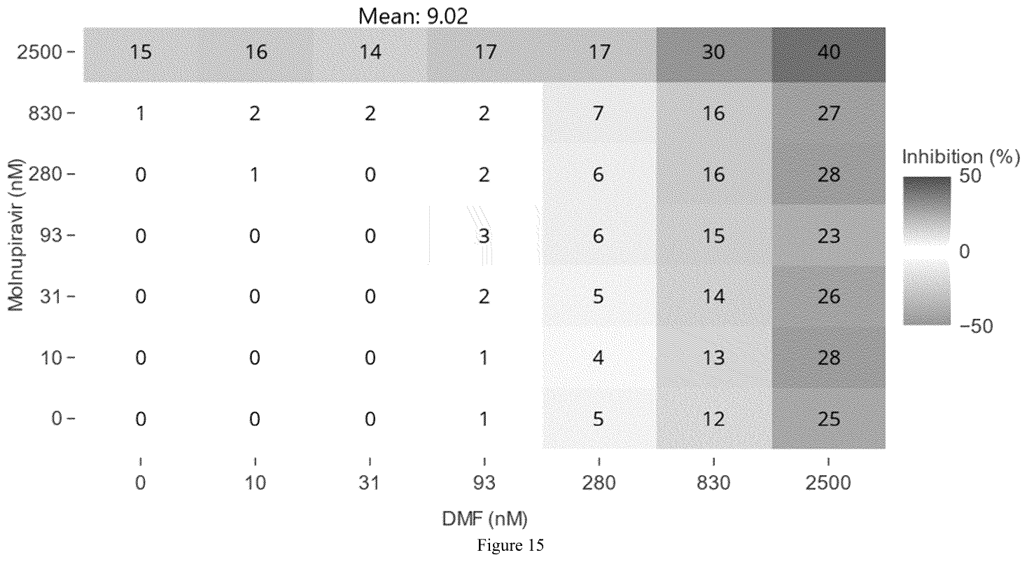

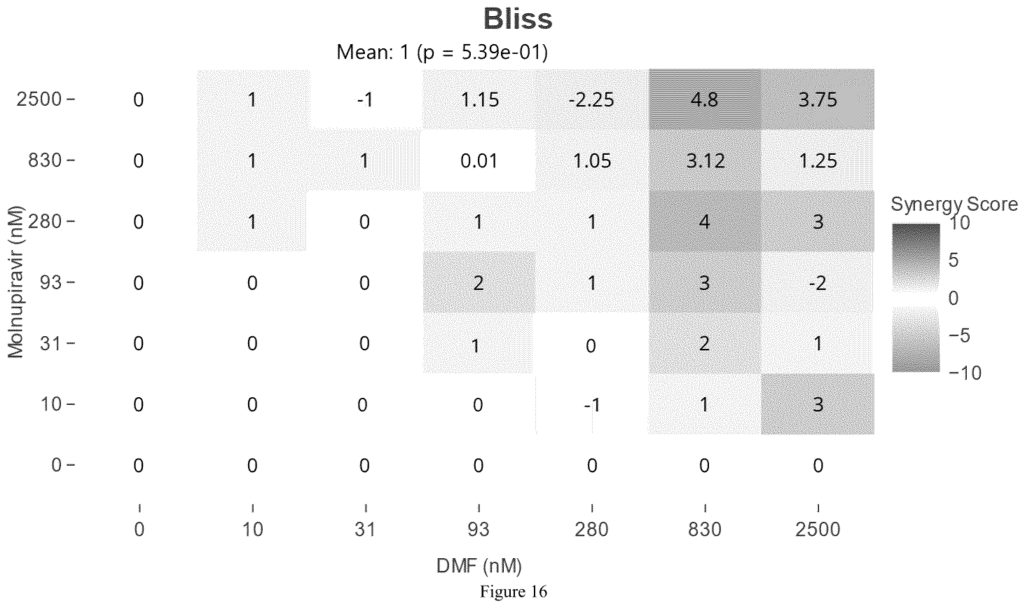

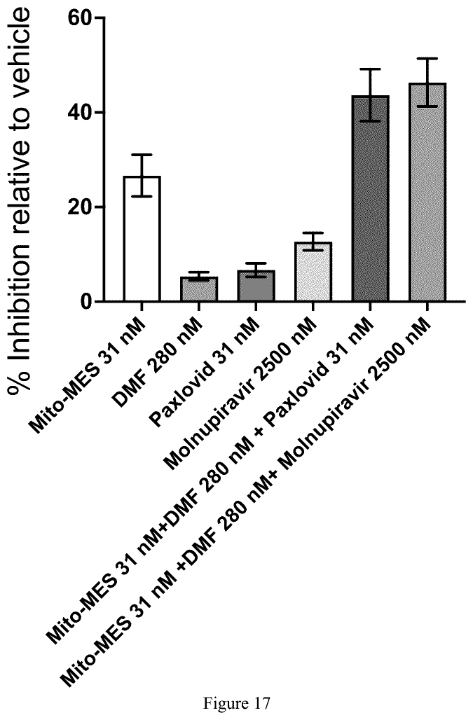

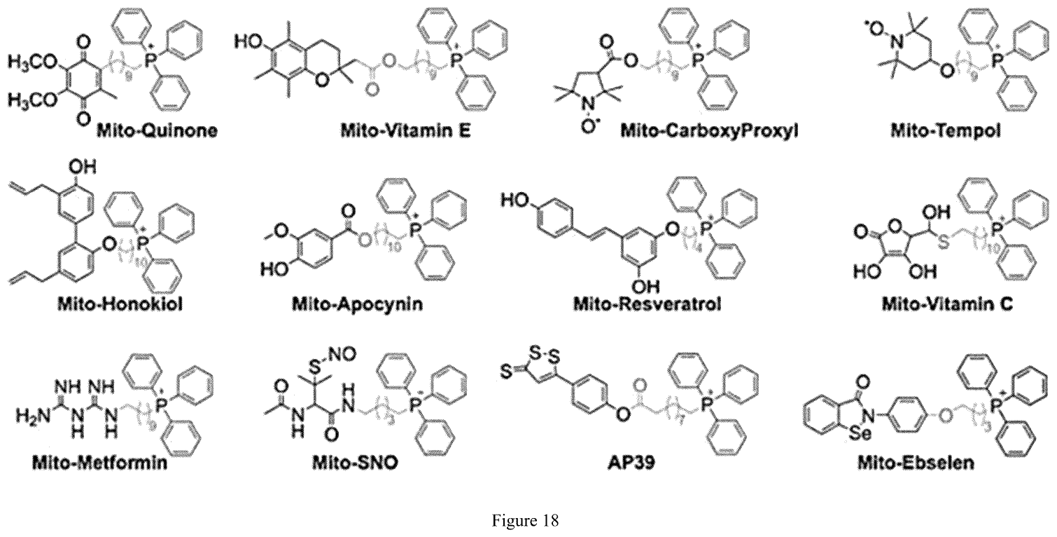

We then performed a dose response matrix (FIG. 15) and synergy distribution analysis (FIG. 16) of DMF and molnupiravir in hACE2-A549 cells infected and treated as shown. We used BLISS analysis to determine whether the drugs interact at concentrations 1 nM-2.5 μM. The null hypothesis is that the drugs are additive. A total synergy score from −10 to 10 suggests that the interaction between two drugs is likely to be additive. A mean synergy score 1.0 was obtained suggesting that the DMF and molnupiravir have additive antiviral effects.

### Nrf2 Agonists, Mito-MES and DMF, Complement Paxlovid™ and Molnupiravir In Vitro to Attenuate SARS-CoV-2 Replication in Lung Epithelial Cells.

We then determined whether the combination of 3 drugs together (Mito-MES plus DMF plus Paxlovid™ or molnupiravir) have additive effects at therapeutic concentrations that are at least one third of the mean value of IC50 for each drug. We used the same experimental system for all experiments, the h-ACE2 A549 cell. As outlined in FIG. 1 to FIG. 15 the IC50 of Mito-MES is 144 nM, the IC50 of Paxlovid™ is 177 nM, the IC50 of DMF is 16.711M and the IC50 of molnupiravir is 31 μM. Thus, we chose concentrations of Mito-MES of 31 nM, DMF of 280 nM, Paxlovid™ of 31 nM and molnupiravir 2.5 μM so that each drug alone gave antiviral effect of <30% compared to DMSO vehicle control. This approached allowed determination of any additive antiviral effects or synergistic (higher than additive). In single layer high ACE2 A549 lung epithelial cells, the combination of Mito-MES plus DMF plus Paxlovid™ gave higher antiviral effect compared to Paxlovid™ alone. The combination of Mito-MES plus DMF plus molnupiravir gave higher antiviral effect compared to molnupiravir alone. See FIG. 14.

In view of the results herein, Nrf2 agonists such Mito-MES and/or DMF may be used in conjunction with antivirals such as Paxlovid™ and molnupiravir as a combination therapeutic to, not only inhibit or reduce viral replication, but also treat, inhibit, or reduce cytotoxic injury, aberrant host inflammatory responses, and the progression of lung injury caused by viral infection by, e.g., coronaviruses such as SARS-CoV-2.

Paxlovid™ is a therapeutic comprising nirmatrelvir and ritonavir. Nirmatrelvir is a 3C-like protease (3CLpro) inhibitor, ritonavir is a protease inhibitor, and molnupiravir is an N⁴-hydroxycytidine nucleoside.

Thus, the present invention is directed to methods, compositions, and kits employing (a) one or more Nrf2 agonists; and (b) one or more 3CLpro inhibitors, one or more protease inhibitors, and/or one or more N⁴-hydroxycytidine nucleosides, as a combination therapeutic for treating, inhibiting, or reducing a viral infection, cytotoxic injury caused by a viral infection, an aberrant host inflammatory response caused by a viral infection, and/or lung injury caused by a viral infection.

In some embodiments, the Nrf2 agonist is a TPP Compound, a mitochondrial targeted antioxidant, dimethyl fumarate (DMF), antcin C, baicalein, butein, carthamus red, curcumin, diallyl disulfide, ellagic acid, gastrodin, ginsenoside Rg1, ginsenoside Rg3, glycyrrhetinic acid, hesperidin, isoorientin, linalool, lucidone, lutein, lycopene, mangiferin, naringenin, oleanolic acid, oroxylin A, oxyresveratrol, paeoniflorin, phloretin, puerarin, quercetin, resveratrol, S-allylcysteine, salvianolic acid B, sauchinone, schisandrin B, sulforaphane, tungtungmadic acid, withaferin A, or alpha-lipoic acid. In some embodiments, the Nrf2 agonist is Mito-MES and/or DMF.

In some embodiments, the 3CLpro inhibitor is a nirmatrelvir compound. In some embodiments, the nirmatrelvir compound is nirmatrelvir. In some embodiments, the protease inhibitor is a ritonavir compound, atazanavir (Reyataz), darunavir (Prezista), fosamprenavir (Lexiva), indinavir (Crixivan), lopinavir/ritonavir (KALETRA), nelfinavir (Viracept), saquinavir (Invirase), tipranavir (Aptivus), atazanavir/cobicistat (Evotaz), or darunavir/cobicistat (Prezcobix). In some embodiments, the protease inhibitor is a ritonavir compound. In some embodiments, the ritonavir compound is ritonavir. In some embodiments, the N⁴-hydroxycytidine nucleoside is a nucleoside as described in U.S. Pat. No. 9,809,616, U.S. Ser. No. 10/874,683, U.S. Ser. No. 11/147,826, U.S. Ser. No. 11/197,882, U.S. Ser. No. 11/312,743, U.S. Ser. No. 11/331,331, US20200276219, or US20220185840. In some embodiments, the N⁴-hydroxycytidine nucleoside is molnupiravir.

### Kits

In some embodiments, the present invention provides kits comprising (a) one or more Nrf2 agonists; and (b) one or more 3CLpro inhibitors, one or more protease inhibitors, and/or one or more N⁴-hydroxycytidine nucleosides, packaged together with one or more reagents or drug delivery devices for preventing, inhibiting, reducing, or treating a viral infection in a subject. In some embodiments, the Nrf2 agonists, the 3CLpro inhibitors, the protease inhibitors, and/or the N⁴-hydroxycytidine nucleosides are provided as unit dosage forms, packaged together as a pack and/or in drug delivery device, e.g., a pre-filled syringe.

In some embodiments, the kits include a carrier, package, or container that may be compartmentalized to receive one or more containers, such as vials, tubes, and the like. In some embodiments, the kits optionally include an identifying description or label or instructions relating to its use. In some embodiments, the kits include information prescribed by a governmental agency that regulates the manufacture, use, or sale of compounds and compositions as contemplated herein.

### Compositions

Compositions, including pharmaceutical compositions, comprising, consisting essentially of, or consisting of (a) one or more Nrf2 agonists; and (b) one or more 3CLpro inhibitors, one or more protease inhibitors, and/or one or more N⁴-hydroxycytidine nucleosides are contemplated herein. In these embodiments, the (a) one or more Nrf2 agonists; and the (b) one or more 3CLpro inhibitors, one or more protease inhibitors, and/or one or more N⁴-hydroxycytidine nucleosides are combined in a single composition. That is, at least one of (a) and at least one of (b) are combined in a single composition.

The term “pharmaceutical composition” refers to a composition suitable for pharmaceutical use in a subject. A composition generally comprises an effective amount of an active agent and a diluent and/or carrier. A pharmaceutical composition generally comprises a therapeutically effective amount of an active agent and a pharmaceutically acceptable carrier.

As used herein, an “effective amount” refers to a dosage or amount sufficient to produce a desired result. The desired result may comprise an objective or subjective change as compared to a control in, for example, in vitro assays, and other laboratory experiments. As used herein, a “therapeutically effective amount” refers to an amount that may be used to treat, prevent, or inhibit a given disease or condition in a subject as compared to a control, such as a placebo. Again, the skilled artisan will appreciate that certain factors may influence the amount required to effectively treat a subject, including the degree of the condition or symptom to be treated, previous treatments, the general health and age of the subject, and the like. Nevertheless, effective amounts and therapeutically effective amounts may be readily determined by methods in the art.

In some embodiments, the one or more Nrf2 agonists is administered daily. In some embodiments, the one or more Nrf2 agonists is administered multiple times per day, e.g., twice daily. In some embodiments, the one or more Nrf2 agonists is administered orally, subcutaneously, or intravenously, preferably orally. In some embodiments, the administration of the one or more Nrf2 agonists is before, during, and/or after the subject was exposed or likely exposed to a given virus such as a coronavirus. In some embodiments, the administration of the one or more Nrf2 agonists occurs for at least 1, 2, 3, 4, 5, 6, 7, 8, 9, 10, 11, 12, 13, or 14 days, after the exposure or likely exposure to the given virus. In some embodiments, the administration of the one or more Nrf2 agonists occurs for at least 1-10 days after the exposure or likely exposure to the given virus.

In some embodiments, the amount of the one or more Nrf2 agonist that is administered to a subject is about 0.02-8.0 mg/kg, about 0.15-8.0 mg/kg, about 4.0-8.0 mg/kg, about 0.01-4.0 mg/kg, about 0.1-4.0 mg/kg, or about 2.0-4.0 mg/kg weight of the subject. In some embodiments, the amount of the one or more Nrf2 agonist that is administered to a subject is about 1-480 mg, about 10-480 mg, about 240-480 mg, about 0.5-240 mg, about 5-240 mg, or about 120-240 mg.

In some embodiments, the amount of the TPP Compound administered to a subject is about 0.05 mg/kg to about 15 mg/kg, preferably about 0.2 mg/kg to about 1.5 mg/kg, or more preferably about 0.3 mg/kg to about 0.7 mg/kg weight of the subject. In some embodiments, the amount of the TPP Compound administered to a subject is about 1-1000 mg, 5-100 mg, 10-80 mg, or 20-40 mg, and preferably about 20 mg. In some embodiments, the amount of the TPP Compound administered to a subject is 1 mg, mg, 10 mg, 15 mg, 20 mg, 25 mg, 30 mg, 35 mg, 40 mg, 45 mg, 50 mg, 55 mg, 60 mg, mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg.

In some embodiments, about 0.05 mg/kg to about 15 mg/kg, preferably about 0.2 mg/kg to about 1.5 mg/kg, or more preferably about 0.3 mg/kg to about 0.7 mg/kg of Mito-MES per weight of the subject is administered to the subject. In some embodiments, about 1-1000 mg, 5-100 mg, 10-80 mg, or 20-40 mg, preferably about 20 mg of Mito-MES is administered to a subject. In some embodiments, 1 mg, 5 mg, 10 mg, 15 mg, 20 mg, 25 mg, 30 mg, 35 mg, 40 mg, 45 mg, 50 mg, 55 mg, 60 mg, mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg of Mito-MES is administered to a subject.

In some embodiments, an amount of DMF of about 1-480 mg, about 10-480 mg, about 240-480 mg, about 0.5-240 mg, about 5-240 mg, or about 120-240 mg is administered to a subject. In some embodiments, an amount of DMF of 1 mg, 5 mg, mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, or 480 mg is administered to a subject. In some embodiments, the amount of the one or more TPP Compounds to the amount of DMF is about 1:10.

In some embodiments, the one or more 3CLpro inhibitors, the one or more protease inhibitors, and/or the one or more N⁴-hydroxycytidine nucleosides is administered daily. In some embodiments, the one or more 3CLpro inhibitors, the one or more protease inhibitors, and/or the one or more N⁴-hydroxycytidine nucleosides is administered multiple times per day, e.g., twice daily. In some embodiments, the one or more 3CLpro inhibitors, the one or more protease inhibitors, and/or the one or more N⁴-hydroxycytidine nucleosides is administered orally, subcutaneously, or intravenously, preferably orally. In some embodiments, the administration of the one or more 3CLpro inhibitors, the one or more protease inhibitors, and/or the one or more N⁴-hydroxycytidine nucleosides is before, during, and/or after the subject was exposed or likely exposed to a given virus such as a coronavirus. In some embodiments, the administration of the one or more 3CLpro inhibitors, the one or more protease inhibitors, and/or the one or more N⁴-hydroxycytidine nucleosides occurs for at least 1, 2, 3, 4, 5, 6, 7, 8, 9, 10, 11, 12, 13, or 14 days, after the exposure or likely exposure to the given virus. In some embodiments, the administration of the one or more 3CLpro inhibitors, the one or more protease inhibitors, and/or the one or more N⁴-hydroxycytidine nucleosides occurs for at least 1-10 days after the exposure or likely exposure to the given virus. In some embodiments, the administration of the one or more 3CLpro inhibitors, the one or more protease inhibitors, and/or the one or more N⁴-hydroxycytidine nucleosides occurs for up to about 5 days upon exhibiting symptoms of having a viral infection.

The dosages of the one or more 3CLpro inhibitors, the one or more protease inhibitors, and/or the one or more N⁴-hydroxycytidine nucleosides depends on the given agent selected. In some embodiments, the dosage of the one or more 3CLpro inhibitors, the one or more protease inhibitors, and/or the one or more N⁴-hydroxycytidine nucleosides will be up to the dose usually prescribed for the given agent. For example, for an adult subject, the daily dose of a ritonavir compound, e.g., ritonavir, is up to 200 mg. As another example, for an adult subject, the daily dose of a nirmatrelvir compound, e.g., nirmatrelvir, is up to 600 mg. For an adult subject, the daily dose of a N⁴-hydroxycytidine nucleoside, e.g., molnupiravir, is up to 1600 mg.

### Exemplary Amounts in Compositions, Kits, and Unit Dosages

In some embodiments, the amount of the one or more Nrf2 agonist is about 1— 480 mg, about 10-480 mg, about 240-480 mg, about 0.5-240 mg, about 5-240 mg, or about 120-240 mg.

In some embodiments, the amount of the one or more TPP Compounds is about 1 1000 mg, 5-100 mg, 10-80 mg, or 20-40 mg, preferably about 20 mg. In some embodiments, the amount of the one or more TPP Compounds is 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg.

In some embodiments, the amount of Mito-MES is 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg.

In some embodiments, the amount of DMF is about 1-480 mg, about 10-480 mg, about 240-480 mg, about 0.5-240 mg, about 5-240 mg, or about 120-240 mg is used or provided. In some embodiments, the amount of DMF is 1 mg, 5 mg, 10 mg, mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, or 480 mg. In some embodiments, the amount of the one or more TPP Compounds to the amount of DMF is about 1:10.

In some embodiments, the amount of the one or more 3CLpro inhibitors, the one or more protease inhibitors, and/or the one or more N⁴-hydroxycytidine nucleosides is up to the amount the given agent is usually provided. For example, the amount of a ritonavir compound, e.g., ritonavir, may be up to 200 mg, preferably up to 100 mg. As another example, the amount of a nirmatrelvir compound, e.g., nirmatrelvir, may be up to 600 mg, preferably up to 300 mg. The amount of a N⁴-hydroxycytidine nucleoside, e.g., molnupiravir, may be up to 1600 mg, preferably up to 800 mg.

Combinations of (a) one or more Nrf2 agonists; and (b) one or more 3CLpro inhibitors, one or more protease inhibitors, and/or one or more N⁴-hydroxycytidine nucleosides may be administered, preferably in the form of pharmaceutical compositions, to a subject. Preferably the subject is mammalian, more preferably, the subject is human. Preferred pharmaceutical compositions are those comprising a combination of (a) one or more Nrf2 agonists; and (b) one or more 3CLpro inhibitors, one or more protease inhibitors, and/or one or more N⁴-hydroxycytidine nucleoside in a therapeutically effective amount and a pharmaceutically acceptable vehicle.

In some embodiments, the one or more Nrf2 agonists comprises (a) DMF, a TPP Compound (e.g., Mito-MES), or both DMF and a TPP Compound (e.g., Mito-MES); and (b) a ritonavir compound (e.g., ritonavir), a nirmatrelvir compound (e.g., nirmatrelvir), a ritonavir compound (e.g., ritonavir) and a nirmatrelvir compound (e.g., nirmatrelvir). In some embodiments, the one or more Nrf2 agonists comprises (a) DMF, a TPP Compound (e.g., Mito-MES), or both DMF and a TPP Compound (e.g., Mito-MES); and (b) a N⁴-hydroxycytidine nucleoside (e.g., molnupiravir).

Pharmaceutical compositions may be formulated for the intended route of delivery and administered to subjects accordingly using methods in the art. Suitable routes of delivery include auricular, buccal, conjunctival, cutaneous, dental, electro-osmosis, endocervical, endosinusal, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, respiratory, retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal delivery routes. It will be appreciated that the preferred route of administration and pharmaceutical formulation will vary with the condition and age of the subject, the nature of the condition to be treated, the therapeutic effect desired, and the particular combination therapy used.

Pharmaceutical compositions may include one or more of the following: a pharmaceutically acceptable vehicle, pH buffered solutions, adjuvants (e.g., preservatives, wetting agents, emulsifying agents, and dispersing agents), liposomal formulations, nanoparticles, dispersions, suspensions, or emulsions, as well as sterile powders for reconstitution into sterile injectable solutions or dispersions. The compositions and formulations may be optimized for increased stability and efficacy using methods in the art. See, e.g., Carra et al., (2007) Vaccine 25:4149-4158.

As used herein, a “pharmaceutically acceptable vehicle” or “pharmaceutically acceptable carrier” are used interchangeably and refer to solvents, dispersion media, coatings, antibacterial and antifungal agents, isotonic and absorption delaying agents, and the like, that are compatible with pharmaceutical administration and comply with the applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable vehicles include those known in the art. See, e.g., Remington: The Science and Practice of Pharmacy 20^{th} ed (2000) Lippincott Williams & Wilkins, Baltimore, MD.

A “pharmaceutically acceptable solvate” refers to a solvate form of a specified compound that retains the biological effectiveness of such compound. Examples of solvates include compounds of the invention in combination with water, isopropanol, ethanol, methanol, dimethyl sulfoxide, ethyl acetate, acetic acid, ethanolamine, or acetone. Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of compounds of formulas I and II are within the scope of the invention. It will also be appreciated by those skilled in organic chemistry that many organic compounds can exist in more than one crystalline form. For example, crystalline form may vary from solvate to solvate. Thus, all crystalline forms of the compounds described herein and the pharmaceutically acceptable solvates thereof are contemplated herein.

The term “pharmaceutically acceptable salts” refers to salt forms that are pharmacologically acceptable and substantially non-toxic to the subject being treated with the compound of the invention. Pharmaceutically acceptable salts include conventional acid-addition salts or base-addition salts formed from suitable non-toxic organic or inorganic acids or inorganic bases. Exemplary acid-addition salts include those derived from inorganic acids such as hydrochloric acid, hydrobromic acid, hydroiodic acid, sulfuric acid, sulfamic acid, phosphoric acid, and nitric acid, and those derived from organic acids such as p-toluenesulfonic acid, methanesulfonic acid, ethane-disulfonic acid, isethionic acid, oxalic acid, p-bromophenylsulfonic acid, carbonic acid, succinic acid, citric acid, benzoic acid, 2-acetoxybenzoic acid, acetic acid, phenylacetic acid, propionic acid, glycolic acid, stearic acid, lactic acid, malic acid, tartaric acid, ascorbic acid, maleic acid, hydroxymaleic acid, glutamic acid, salicylic acid, sulfanilic acid, and fumaric acid. Exemplary base-addition salts include those derived from ammonium hydroxides (e.g., a quaternary ammonium hydroxide such as tetramethylammonium hydroxide), those derived from inorganic bases such as alkali or alkaline earth-metal (e.g., sodium, potassium, lithium, calcium, or magnesium) hydroxides, and those derived from non-toxic organic bases such as basic amino acids.

A “pharmaceutically acceptable prodrug” is a compound that may be converted under physiological conditions or by solvolysis to the specified compound or to a pharmaceutically acceptable salt of such compound. A “pharmaceutically active metabolite” refers to a pharmacologically active product produced through metabolism in the body of a specified compound or salt thereof. Prodrugs and active metabolites of a compound may be identified using routine techniques known in the art. See, e.g., Bertolini, G, et al., (1997) J Med Chem 40:2011-2016; Shan, D, et al., J Pharm Sci, 86(7):765-767; Bagshawe K., (1995) Drug Dev Res 34:220-230; Bodor, N, (1984) Advances in Drug Res 13:224-331; Bundgaard, H, Design of Prodrugs (Elsevier Press, 1985) and Larsen, I K, Design and Application of Prodrugs, Drug Design and Development (Krogsgaard-Larsen, et al., eds., Harwood Academic Publishers, 1991).

The pharmaceutical compositions may be provided in dosage unit forms. As used herein, a “dosage unit form” refers to physically discrete units suited as unitary dosages for the subject to be treated; each unit containing a predetermined quantity of (a) one or more Nrf2 agonists; and (b) one or more 3CLpro inhibitors, one or more protease inhibitors, and/or one or more N⁴-hydroxycytidine nucleoside calculated to produce the desired therapeutic effect in association with the required pharmaceutically acceptable carrier. The specification for the dosage unit forms of the invention are dictated by and directly dependent on the unique characteristics of the given combination of (a) one or more Nrf2 agonists; and (b) one or more 3CLpro inhibitors, one or more protease inhibitors, and/or one or more N⁴-hydroxycytidine nucleoside and desired therapeutic effect to be achieved, and the limitations inherent in the art of compounding such an active compound for the treatment of individuals.

Toxicity and therapeutic efficacy of combinations of (a) one or more Nrf2 agonists; and (b) one or more 3CLpro inhibitors, one or more protease inhibitors, and/or one or more N⁴-hydroxycytidine nucleosides according to the instant invention can be determined using cell cultures and/or experimental animals and pharmaceutical procedures in the art. For example, one may determine the lethal dose, LC₅₀ (the dose expressed as concentration x exposure time that is lethal to 50% of the population) or the LD₅₀ (the dose lethal to 50% of the population), and the ED₅₀ (the dose therapeutically effective in 50% of the population) by methods in the art. The dose ratio between toxic and therapeutic effects is the therapeutic index and it can be expressed as the ratio LD₅₀/ED₅₀. Combinations of (a) one or more Nrf2 agonists; and (b) one or more 3CLpro inhibitors, one or more protease inhibitors, and/or one or more N⁴-hydroxycytidine nucleosides which exhibit large therapeutic indices are preferred. While combinations of (a) one or more Nrf2 agonists; and (b) one or more 3CLpro inhibitors, one or more protease inhibitors, and/or one or more N⁴-hydroxycytidine nucleosides that result in toxic side-effects may be used, care should be taken to design a delivery system that targets such compounds to the site of treatment to minimize potential damage to uninfected cells and, thereby, reduce side-effects.

The data obtained from the cell culture assays and animal studies can be used in formulating a range of dosages for use in humans. Preferred dosages provide a range of circulating concentrations that include the ED₅₀ with little or no toxicity. The dosage may vary depending upon the dosage form employed and the route of administration utilized. Therapeutically effective amounts and dosages of a combination of (a) one or more Nrf2 agonists; and (b) one or more 3CLpro inhibitors, one or more protease inhibitors, and/or one or more N⁴-hydroxycytidine nucleosides can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Additionally, a dosage suitable for a given subject can be determined by an attending physician or qualified medical practitioner, based on various clinical factors.

The following examples are intended to illustrate but not to limit the invention.

### EXAMPLES

### Cells

Human lung carcinoma cells (A549) expressing human angiotensin-converting enzyme 2 (hACE2-A549) cells (NR-53821) were obtained through BEI Resources (Manassas, VA). Normal human bronchial epithelial cells (NHBE) (Cat #CC-2540) were obtained from Lonza (Basel, Switzerland), and all samples were de-identified. Lonza lung samples were obtained from donors ranging between 30-50 years and represented both males and females.

### Viruses

The following reagents were obtained through Biodefense and Emerging Infectious (BEI) Resources of National Institute of Allergy and Infectious Diseases (NIAID), National Institutes of Health (NIH) (Manassas, VA): SARS-CoV-2, isolates 1) hCoV-19/USA/PHC658/2021 (Lineage B.1.617.2; Delta Variant), NR-55611, contributed by Dr. Richard Webby and Dr. Anami Patel, 2) Isolate hCoV-19/USA/MD-HP05285/2021 (Lineage B.1.617.2; Delta Variant), NR-55671, contributed by Andrew S. Pekosz, 3) Isolate hCoV-19/USA/GA-EHC-2811C/2021 (Lineage B.1.1.529; Omicron Variant), NR-56481, contributed by Mehul Suthar.

### Antibodies

Rabbit anti-SARS-CoV-2 nucleocapsid protein (NP) (clone ARC2372, Cat#PIMA536086) was purchased from Thermo Fisher Scientific (Waltham, MA). Rabbit anti-SARS-CoV-2 NP (polyclonal, Cat #200401A50) was purchased from Rockland Immunochemicals (Pottstown, PA). Rabbit anti-SARS-CoV-2 (2019-nCoV) Spike 51 (clone 007, Cat #40150-R007) was purchased from Sino Biologicals (Beijing, China). Mouse anti-SARS-CoV/SARS-CoV-2 (COVID-19) Spike (clone 1A9, Cat#GTX632604) was purchased from Genetex (Irvine, CA). The following secondary antibodies were purchased: goat anti-mouse Horseradish Peroxidase (HRP) (Cat#A16066), goat anti-rabbit HRP (Cat #PI31460) were purchased from Thermo Fisher Scientific.

### Immunoassays

The following enzyme-linked immunosorbent assays (ELISA) were purchased: Anti-SARS-CoV-2 Nucleocapsid Protein Sandwich ELISA Kit (Cat #GTX535824) was purchased from Genetex (Irvine, CA).

### Other Reagents

The following materials were purchased from MilliporeSigma (Burlington, MA): Dimethyl fumarate (DMF) (Cat #242926-25G), Dimethyl Sulfoxide (DMSO) (Cat#472301-100ML), Triton X-100 (Cat #T8787-50ML).

The following materials were purchased from Thermo Fisher Scientific (Waltham, MA): Corning™ Costar™ 96-Well, Cell Culture-Treated, Flat-Bottom Microplate (Cat #07-200-91), Dulbecco's modified eagle medium (DMEM) (Cat #10-569-044), Dulbecco's modified eagle medium (DMEM) low glucose (Cat #11054-020), Hanks buffered salt solution (HBSS) (Cat #14-025-092), Hoechst 33342 (Cat #H3570), Hygromycin (Cat #10-687-010), L-Glutamine 200 mM (Cat #25-030-081), Eagle's minimum essential medium (MEM) (Cat #MT10009CV), non-essential amino acids 100X (Cat #SH3023801), Pierce™ Bicinchoninic Acid Assay (BCA) protein assay kit (Cat #PI23227),Penicillin/Streptomycin 100X (Cat #15-140-122), Sodium Pyruvate 100 mM (Cat #11-360-070), sterile nylon 40 μm Filter (Cat #07-201-430), 3,3′,5,5′-tetramethylbenzidine (TMB) peroxidase substrate (Cat #N³⁰¹), T-PERTH tissue protein extraction reagent (Cat #PI78510), and trypsin-ethylenediamine tetraacetic acid (EDTA) (0.25% w/v).

The following reagents were purchased from Cayman Chemical (Ann Arbor, MI): Mitoquinone mesylate (Mito-MES) (Cat #29317), Mitoquinol mesylate (Cat#89950).

Fetal Bovine Serum (FBS) (Cat #100-500) was purchased from GeminiBio (West Sacramento, CA). Costar 6.5 mm Transwell® 0.4 μm pore polyester membrane inserts (Cat #38024), PneumaCult Ex-Plus media (Cat #05040) and PneumaCult Air-Liquid Interface (ALI)-S media (Cat #05050) were purchased from Stem Cell Technologies (Vancouver, Canada). XTT Cell Proliferation Assay Kit (Cat #30-1011K) was purchased from ATCC. Stop Solution 2N Sulfuric Acid (Cat #DY994) was purchased from Bio-Techne (Minneapolis, MN). Intracellular Staining Permeabilization Wash Buffer (10x) (Cat #421002) were purchased from BioLegend (San Diego, CA). 32% w/v Paraformaldehyde (PFA) aqueous solution (Cat #15714-1L) was purchased from Electron Microscopy Sciences (Hatfield, PA). Black 96 well μCLEAR® cell culture microplate (Cat #655090) was purchased from Greiner Bio-One (Kremsmiinster, Austria).

Nirmatrelvir (PF-07321332) (Cat. No.: HY-138687) and Molnupiravir (Synonyms: EIDD-2801; MK-4482) (Cat. No.: HY-135853) were purchased from MedChemExpress (New Jersey, NY).

### Cell Cultures

Cells were maintained at 37° C. and 5% CO₂ in DMEM or MEM supplemented with 10% (v/v) FBS, penicillin (100 units/ml), and streptomycin (100 μg/ml) (1X P/S). h-ACE2 A549 cells were maintained at 37° C. and 5% CO₂ in DMEM supplemented with 10% (v/v) FBS and blasticidin.

### Human Bronchial Epithelial Cells (HBEC) Air-Liquid Interface Cultures (Upper Airway ALI Cultures)

24-well 6.5 mm transwells with 0.4 μm pore polyester membrane inserts were coated with collagen type I dissolved in cell culture grade water at a ratio of 1:10. 100 μl was added to each transwell and allowed to air dry. HBEC were seeded at 100,000 cells per well directly onto collagen-coated transwells and allowed to grow in the submerged phase of culture for 4-5 days with 500 μl media in the basal chamber and 200 μl media in the apical chamber. ALI cultures were then established and cultured with only 500 μl media in the basal chamber, and cultures were infected with SARS-CoV-2 as indicated. Media was changed every other day and cultures were maintained at 37° C. and 5% CO₂.

### SARS-CoV-2 Infection

All studies involving live virus were conducted at the UCLA Biosafety Level 3 (BSL3) high-containment facility with appropriate institutional biosafety approvals. SARS-CoV-2 was passaged once in Vero-E6 cells and viral stocks were aliquoted and stored at −80° C. Virus titer was measured in Vero-E6 cells by median tissue culture infectious dose (TCID₅₀) assay. Cell cultures in 96 well plates and ALI cultures were infected with SARS-CoV-2 viral inoculum [Multiplicity of infection (MOI) of 0.1 or at least 1 for ALI; 100 μl/well] prepared in media. For infection of hACE2-A549 cells in 96 well plates an MOI of 0.5 or 1 and 50 μl/well was used. For mock infection, conditioned media (50 or 10011.1/well) alone was added.

### Assessment of SARS-CoV-2 Infection Among Different Cell Culture Systems

SARS-CoV-2 infection was assessed by independent experiments that determined the total amount of secreted or intracellular SARS-CoV-2. The intracellular content of the SARS-CoV-2 nucleocapsid protein (NP) was assessed using ELISA. Protein extract concentration was quantified using the Pierce BCA Protein Assay, according to manufacturer instructions (Thermo Fisher Scientific, Waltham, MA). In ELISA experiments the intracellular SARS-CoV-2 NP protein content (pg) was normalized by the total cellular amount of protein within each experimental well (pg of viral protein perm of total protein). The secreted amount of live SARS-CoV-2 in cell culture supernatant of infected cell cultures was assessed using viral titer. Percent infection was quantified as ((Infected cells/Total cells)−Background)*100 and the vehicle control was then set to 100% infection for analysis. The half maximal inhibitory concentration (IC50) for each experiment were determined using the Prism (GraphPad Holdings, San Diego, CA) software.

### Viral Titers

Infectious titers were quantified by limiting dilution titration using Vero-E6 cells. Briefly, Vero-E6 cells were seeded in 96-well plates at 5,000 or 10,000 cells/well. The next day, SARS-CoV-2-containing supernatant was applied at serial 10-fold dilutions ranging from 10⁻¹ to 10-8 and, after 3-5 days, viral cytopathic effect (CPE) was assessed by microscopy or by determination of the intracellular SARS-CoV-2 NP using ELISA. TCID₅₀/ml was calculated using the Reed-Muench method.

### In-Cell SARS-CoV-2 ELISA

To independently establish detection of SARS-CoV-2 infection using a more quantitative method to assess viral titer (not based on microscopy), we utilized in-cell SARS-CoV-2 ELISA based on intracellular detection of the SARS-CoV-2 NP protein. or 10,000 Vero-E6 cells were seeded in 96 well plates in 100 μl. The next day, the cells were inoculated with 10 μl of a 10-fold titration series of SARS-CoV-2. Two to three days later, SARS-CoV-2 NP protein staining was assessed using an anti-SARS-CoV-2 NP protein antibody. Cells were fixed by adding 100 μl 8% (v/v) PFA to 100 μl of medium (final 4% solution) and 30 min of room temperature incubation. Medium was then discarded and the cells permeabilized for 5 min at room temperature by adding 100 μl of Intracellular Staining Permeabilization Wash Buffer (BioLegend). Cells were then washed with PBS and stained with 1:10,000 (anti-NP antibody clone ARC2372) in permeabilization buffer at 37° C. After 1 hour, the cells were washed three times with washing buffer before a secondary anti-mouse or anti-rabbit antibody conjugated with HRP was added (1:20,000) and incubated for 1 hour at 37° C. Following three times of washing, the 3,3′,5,5′-tetramethylbenzidine (TMB) peroxidase substrate was added. After 5 min light-protected incubation at room temperature, reaction was stopped using M H2SO4. The optical density (OD) was recorded at 450 nm and baseline corrected for 620 nm using the Biotek microplate reader (Agilent Technologies, Santa Clara, CA).

### Drug Treatments

A concentration of Mito-MES between 10-1000 nM has been shown to be physiologically relevant, efficacious and non-cytotoxic in human mammalian cells. A concentration of DMF between 0.1-100 μM has been shown to be physiologically relevant, efficacious and non-cytotoxic in human mammalian cells. The antiviral activity of Nrf2 agonists was evaluated in h-ACE2 A549 and HBEC ALI cell cultures. All SARS-CoV-2 studies were performed in biological triplicate. Cultured cells were incubated separately with Mito-MES or DMF or Paxlovid™ or molnupiravir. All drug concentrations were between 10 nM to 1001.1M as shown in the Figures. The concentration of DMSO vehicle control was maintained constant at 0.1% v/v for all treatments. Drug effects were measured relative to vehicle controls in vitro. Unless stated, cells ALI cultures were pretreated for at 1-3 hours with the indicated treatments (Mito-MES, DMF, Paxlovid™, molnupiravir) or vehicle control. The cells were then washed, infected with SARS-CoV-2 for 2 hours (hrs), the virus was removed, and the treatments were added back.

### Quantitative Drug Combination Analysis

Paxlovid™, molnupiravir and Mito-MES were added to hACE2-A549 cells in 96-well assay plates in a matrix that combined up to 8 concentrations of drugs (up to 2.5 μM) in up to 0.1% DMSO, resulting in a matrix of up to 64 drug-x drug-y concentration pairs monitoring viral infection. Each combination was independently repeated at least three times as technical duplicates in each biological replicate. Sample well infection was normalized to aggregated 0.1% DMSO plate control wells and expressed as percent of inhibition. Synergy between drug combinations was determined by the BLISS independence model, to quantitatively assess drug interaction patterns within the drug-drug combination matrix. The BLISS expectation (E) for a combined response was calculated by E=(A+B)−(A×B) where A and B are the fractional inhibition of SARS-CoV-2 infection of drug A and drug B at a given dose. The difference between the BLISS expectation a-d the observed inhibition of SARS-CoV-2 infection for the combination of drug A and drug B at the same dose is the BLISS value. BLISS values between 0 and 10 indicate that the combination is additive (as expected for independent pathway effects); BLISS value >20 indicates activity greater than additive (synergy); and BLISS value <0 indicates the combination is less than additive (antagonism). The software SynergyFinder 3.0 was used for an interactive analysis and consensus interpretation of multi-drug synergies using methods in the art.

### Assessment of Cell Cytotoxicity

To measure cell viability to determine if there was any treatment-induced cytotoxicity, uninfected cells were plated and treated with the same compound dilutions used for the in vitro efficacy studies. As above, 0.01% DMSO-treated cells served as the 0% cytotoxicity control. After 24-48 hours (hrs), cell viability was measured on a Synergy 2 Biotek microplate reader (Agilent Technologies, Santa Clara, CA) via the XTT Cell Proliferation Assay Kit according to the manufacturer's protocol (ATCC, Manassas, VA). Similar data were obtained in three independent experiments. LDH cytotoxicity assay was also used to assess cytotoxicity in cell culture supernatant according to the manufacturer's protocol (Sigma-Aldrich).

### REFERENCES

The following references are herein incorporated by reference in their entirety with the exception that, should the scope and meaning of a term conflict with a definition explicitly set forth herein, the definition explicitly set forth herein controls:
Vandyck K, Deval J. Considerations for the discovery and development of 3-chymotrypsin-like cysteine protease inhibitors targeting SARS-CoV-2 infection. Curr Opin Virol. 2021; 49:36-40.
Girardin F, Manuel 0, Marzolini C, Buclin T. Evaluating the risk of drug-drug interactions with pharmacokinetic boosters: the case of ritonavir-enhanced nirmatrelvir to prevent severe COVID-19. Clin Microbiol Infect. 2022.
Sticher Z M, Lu G, Mitchell D G, Marlow J, Moellering L, Bluemling G R, et al. Analysis of the Potential for N (4)-Hydroxycytidine To Inhibit Mitochondrial Replication and Function. Antimicrob Agents Chemother. 2020;64(2).
Zhou S, Hill C S, Sarkar S, Tse L V, Woodburn B M D, Schinazi R F, et al. beta-d-N⁴-hydroxycytidine Inhibits SARS-CoV-2 Through Lethal Mutagenesis But Is Also Mutagenic To Mammalian Cells. J Infect Dis. 2021; 224(3):415-9.
Greenhalgh T, Knight M, A′Court C, Buxton M, Husain L. Management of post-acute covid-19 in primary care. BMJ. 2020;370:m3026.
Pruijssers A J, Denison MR. Nucleoside analogues for the treatment of coronavirus infections. Curr Opin Virol. 2019; 35:57-62.
Huang C, Wang Y, Li X, Ren L, Zhao J, Hu Y, et al. Clinical features of patients infected with 2019 novel coronavirus in Wuhan, China. Lancet. 2020; 395(10223):497-506.
Hammond J, Leister-Tebbe H, Gardner A, Abreu P, Bao W, Wisemandle W, et al. Oral Nirmatrelvir for High-Risk, Nonhospitalized Adults with Covid-19. N Engl J Med. 2022; 386(15):1397-408.
Gottlieb R L, Vaca C E, Paredes R, Mera J, Webb B J, Perez G, et al. Early Remdesivir to Prevent Progression to Severe Covid-19 in Outpatients. N Engl J Med. 2022; 386(4):305-15.
Zhang C, Zhu R, Cao Q, Yang X, Huang Z, An J. Discoveries and developments of CXCR4-targeted HIV-1 entry inhibitors. Exp Biol Med (Maywood). 2020;245(5):477-85.
Jayk Bernal A, Gomes da Silva M M, Musungaie D B, Kovalchuk E, Gonzalez A, Delos Reyes V, et al. Molnupiravir for Oral Treatment of Covid-19 in Nonhospitalized Patients. N Engl J Med. 2022; 386(6):509-20.
Wu B, Chien E Y, Mol C D, Fenalti G, Liu W, Katritch V, et al. Structures of the CXCR4 chemokine GPCR with small-molecule and cyclic peptide antagonists. Science. 2010; 330(6007):1066-71.
Gandhi S, Klein J, Robertson A J, Pena-Hernandez M A, Lin M J, Roychoudhury P, et al. De novo emergence of a remdesivir resistance mutation during treatment of persistent SARS-CoV-2 infection in an immunocompromised patient: a case report. Nat Commun. 2022;13(1):1547.
Wensing A M, van Maarseveen N M, Nijhuis M. Fifteen years of HIV Protease Inhibitors: raising the barrier to resistance. Antiviral Res. 2010; 85(1):59-74.
Halfon P, Locarnini S. Hepatitis C virus resistance to protease inhibitors. J Hepatol. 2011; 55(1):192-206.
Lordan R, Rando H M, Consortium C-R, Greene C S. Dietary Supplements and Nutraceuticals Under Investigation for COVID-19 Prevention and Treatment. ArXiv. 2021.
Ludwig S, Pleschka S, Planz 0, Wolff T. Ringing the alarm bells: signalling and apoptosis in influenza virus infected cells. Cell Microbiol. 2006; 8(3):375-86.
Khan N M, Ahmad I, Haqqi T M. Nrf2/ARE pathway attenuates oxidative and apoptotic response in human osteoarthritis chondrocytes by activating ERK1/2/ELK1-P70S6K-P9ORSK signaling axis. Free Radic Biol Med. 2018; 116:159-71.
Niture S K, Jaiswal A K. Nrf2 protein up-regulates antiapoptotic protein Bcl-2 and prevents cellular apoptosis. J Biol Chem. 2012; 287(13):9873-86.
Braakhuis A J, Nagulan R, Somerville V. The Effect of MitoQ on Aging-Related Biomarkers: A Systematic Review and Meta-Analysis. Oxid Med Cell Longev. 2018; 2018:8575263.
Smith R A, Murphy M P. Animal and human studies with the mitochondria-targeted antioxidant MitoQ. Ann N Y Acad Sci. 2010; 1201:96-103.
Gane E J, Weilert F, Orr D W, Keogh G F, Gibson M, Lockhart M M, et al. The mitochondria-targeted anti-oxidant mitoquinone decreases liver damage in a phase II study of hepatitis C patients. Liver Int. 2010; 30(7):1019-26.
Snow B J, Rolfe F L, Lockhart M M, Frampton C M, O'Sullivan J D, Fung V, et al. A double-blind, placebo-controlled study to assess the mitochondria-targeted antioxidant MitoQ as a disease-modifying therapy in Parkinson's disease. Mov Disord. 2010; 25(11):1670-4.
Rossman M J, Santos-Parker J R, Steward C A C, Bispham N Z, Cuevas L M, Rosenberg H L, et al. Chronic Supplementation With a Mitochondrial Antioxidant (MitoQ) Improves Vascular Function in Healthy Older Adults. Hypertension. 2018; 71(6):1056-63.
Ianevski A, Giri A K, Aittokallio T. SynergyFinder 3.0: an interactive analysis and consensus interpretation of multi-drug synergies across multiple samples. Nucleic Acids Res. 2022.

All scientific and technical terms used in this application have meanings commonly used in the art unless otherwise specified.

As used herein, a “coronavirus” refers to a virus belonging to the family Coronaviridae. In some embodiments, the coronavirus belongs to the subfamily Orthocoronavirinae. In some embodiments, the coronavirus belongs to the genera Alphacoronavirus or Betacoronavirus. In some embodiments, the coronavirus is a human coronavirus. In some embodiments the coronavirus is HCoV-229E, HCoV-NL63, HCoV-0C43, HCoV-HKU1, SARS-CoV, MERS-CoV, or SARS-CoV-2, preferably SARS-CoV, MERS-CoV, or SARS-CoV-2, more preferably SARS-CoV-2. As used herein, “SARS-CoV-2” includes the original strain and its variants (e.g., Alpha (B.1.1.7, Q lineages), Beta (B.1.351), Gamma (P.1), Epsilon (B.1.427, B.1.429), Eta (B.1.525), Iota (B.1.526), Kappa (B.1.617.1), 1.617.3, Mu (B.1.621, B.1.621.1), Zeta (P.2), Delta (B.1.617.2, AY lineages), and Omicron (B.1.1.529, BA lineages), and descendant lineages thereof).

In some embodiments, the virus belongs to the Orthomyxoviridae family, such as an Alphainfluenzavirus, a Betainfluenzavirus, a Gammainfluenzavirus, a Deltainfluenzavirus, an Isavirus, a Quaranj avirus, or a Thogotovirus, more preferably, the virus is an Influenza A virus, an Influenza B virus, an Influenza C virus, or an Influenza D virus, even more preferably the virus is an Influenza A virus of subtype H1N¹, H2N², H3N², H5N¹, H7N⁹, H7N⁷, H1N², H9N², H7N², H7N³, H5N², H10N7, H10N3, or H5N⁸. In some embodiments, the virus is an H1N¹ Influenza A virus.

In some embodiments, the virus belongs to the Picornaviridae family, preferably the virus is an Enterovirus (preferably a human Enterovirus), more preferably the virus is an Enterovirus A, an Enterovirus B, an Enterovirus C, an Enterovirus D, a Rhinovirus A, a Rhinovirus B, or a Rhinovirus C virus, even more preferably the virus is a Rhinovirus A, a Rhinovirus B, or a Rhinovirus C virus, and most preferably the virus is a Rhinovirus A virus such as human rhinovirus HRV16.

In some embodiments, the virus belongs to the Pneumoviridae family, preferably the virus is a Metapneumovirus or a Orthopneumovirus, more preferably the virus is a human Metapneumovirus or a human Orthopneumovirus, even more preferably the virus is a human respiratory syncytial virus, and most preferably the virus is a human metapneumovirus (HMPV), a human respiratory syncytial virus A2 (HRSV-A2), or a human respiratory syncytial virus B1 (HRSV-B1).

As provided herein, “(a) one or more Nrf2 agonists; and (b) one or more 3CLpro inhibitors, one or more protease inhibitors, and/or one or more N⁴-hydroxycytidine nucleoside” are co-administered as a combination therapeutic.

As used herein, “co-administration” refers to the administration of at least two different agents, i.e., a first agent and a second agent to a subject. In some embodiments, the co-administration is concurrent. In embodiments involving concurrent co-administration, the agents may be administered as a single composition, e.g., an admixture, or as two separate compositions. In some embodiments, the first agent is administered before and/or after the administration of the second agent. Where the co-administration is sequential, the administration of the first and second agents may be separated by a period of time, e.g., minutes, hours, or days. Those of skill in the art understand that the formulations and/or routes of administration of the various agents or therapies used may vary. The appropriate dosage for co-administration can be readily determined by one skilled in the art. In some embodiments, when two or more agents are co-administered, the respective agents are administered at lower dosages than appropriate for their administration alone.

As used herein, a “Nrf2 agonist” refers to agonists and activators of nuclear factor erythroid 2—related factor 2 (Nrf2) and the Nrf2 signaling pathway. Exemplary Nrf2 agonists include TPP Compounds, mitochondrial targeted antioxidants, dimethyl fumarate (DMF), antcin C, baicalein, butein, carthamus red, curcumin, diallyl disulfide, ellagic acid, gastrodin, ginsenoside Rg1, ginsenoside Rg3, glycyrrhetinic acid, hesperidin, isoorientin, linalool, lucidone, lutein, lycopene, mangiferin, naringenin, oleanolic acid, oroxylin A, oxyresveratrol, paeoniflorin, phloretin, puerarin, quercetin, resveratrol, S-allylcysteine, salvianolic acid B, sauchinone, schisandrin B, sulforaphane, tungtungmadic acid, withaferin A, and alpha-lipoic acid. In some embodiments, a given Nrf2 agonist is both a TPP Compound and a mitochondrial targeted antioxidant as set forth herein. In some embodiments, the Nrf2 agonist is a mitochondrial targeted antioxidant but not a TPP Compound, e.g., elamipretide, or vice versa.

The structural formula of the triphenylphosphonium moiety (“TPP moiety”) is as follows:

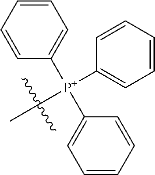 [SMILES: *[P+](c1ccccc1)(c1ccccc1)c1ccccc1]

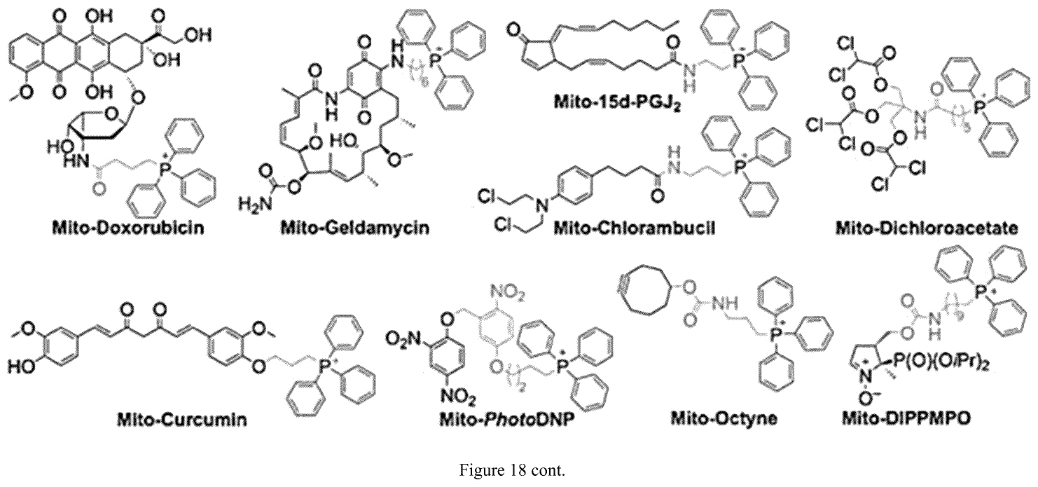

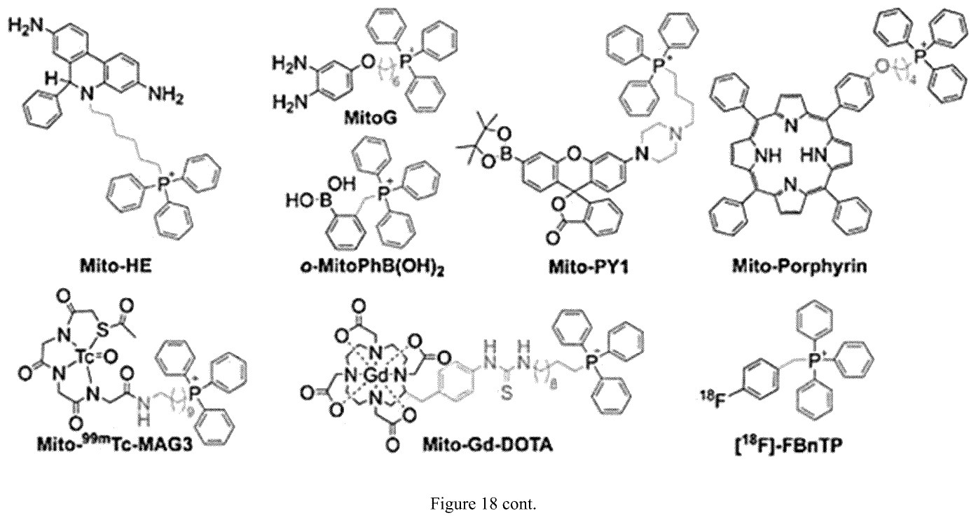

As used herein, a Triphenylphosphonium Compound (“TPP Compound”) refers to a compound that has the TPP moiety as part of its structural formula. Exemplary TPP Compounds include TPP Hydrocarbons and TPP Conjugates. As used herein, “TPP Hydrocarbons” have a saturated or unsaturated, substituted or unsubstituted, branched or unbranched hydrocarbon (HC) group attached to the phosphonium ion. As used herein, “TPP Conjugates” are compounds having a bioactive moiety, i.e., a chemical moiety that exhibits bioactivity by itself, conjugated to the TPP moiety via a linker that is a saturated or unsaturated, substituted or unsubstituted, branched or unbranched hydrocarbon group attached to the phosphonium ion. In some embodiments, the hydrocarbon group is a C₁-C₁₅ alkyl, alkenyl, or alkynyl group. In some embodiments, the hydrocarbon group is a C₁-C₁₀ alkyl, alkenyl, or alkynyl group. In some embodiments, the hydrocarbon group is a C₁-C₁₀ unbranched alkyl group. Exemplary TPP Conjugates include Mito-MES and those set forth in FIG. 18: Mito-Quinone, Mito-Vitamin E, Mito-CarboxyProxyl, Mito-Tempol, Mito-Honokiol, Mito-Apocynin, Mito-Resveratrol, Mito-Vitamin C, Mito-Metformin, Mito-SNO, AP39, Mito-Ebselen, Mito-Doxorubicin, Mito-Geldamycin, Mito-15d-PGJ₂, Mito-Dichloroacetate, Mito-Chlorambucil, Mito-Curcumin, Mito-PhotoDNP, Mito-Octyne, Mito-DIPPMPO, Mito-HE, o-MitoPhB(OH)₂, Mito-PY1, Mito-Porphyrin, Mito-^{99m}Tc-MAG3, Mito-Gd-DOTA, and [¹⁸F]-FBnTP.

Bioactive moieties may be antioxidants (e.g., tocopherol, ubiquinone, thymoquinone, plastoquinone, etc.) and/or Nrf2 agonists (curcumin, resveratrol, etc.). Exemplary bioactive moieties include ubiquinone and derivatives thereof (i.e., compounds having 2,3-dimethoxy-5-methylcyclohexa-2,5-diene-1,4-dione as part of its chemical structure), vitamin E, carboxyproxyl (1-hydroxy-2,2,5,5-tetramethylpyrrolidine-3-carboxylic acid), tempol, honokiol, apocynin, resveratrol, vitamin C, metformin, S-nitrosothiol and compounds containing S-nitrosothiol as part of its chemical structure, dithiolethione, ebselen, doxorubicin, geldamycin, 15d-PGJ2, dichloroacetate, chlorambucil, curcumin, photoDNP (6-[(4-azido-2-nitrophenyl)amino]-N-{6-[(2,4-dinitrophenyl)amino]hexyl}hexanamide), octyne, DIPPMPO, dihydroethidium, phenylboronic acid, dipolar 1,3,6,8-tetrasubstituted pyrene-based compounds (e.g., PY1, PY2, MPY1, MPY2, etc.), porphyrin, 99mTc-MAG3, and Gd-DOTA. A bioactive moiety may be provided as a distinct compound that is not conjugated to the TPP moiety.

As used herein, a “mitochondrial targeted antioxidant” refers to an antioxidant that scavenges reactive oxygen species in mitochondria (“mito-ROS”). Mitochondrial targeted antioxidants include mitoquinone mesylate (10-(4,5-dimethoxy-2-methyl-3,6-dioxo-1,4-cyclohexadienyl) decyl triphenylphosphonium methanesulfonate, derivatives thereof (e.g., the dihydroxy form—mitoquinol mesylate, i.e., 10-(4,5-dimethoxy-2-methyl-3,6-dihydroxy-1,4-cyclohexadienyl) decyl triphenylphosphonium methanesulfonate), mitoquinone and salts thereof (other than methanesulfonate), mitoquinol and salts thereof (other than methanesulfonate), etc.), other mitochondrial targeted antioxidants in the art such as SkQl (Mitotech, S.A.), Elamipretide (Stealth BioTherapeutics), Mito-TEMPO (CAS 1569257-94-8), and those disclosed in the following patents and publications: U.S. Pat. Nos. 8,518,915; 9,192,676; 9,328,130; 9,388,156; US20070161609; US20070225255; US20080161267; 0520100168198; US20160200749; US20180305328; US20190248816; US20190330249; US20190374558; WO2005019232; WO2006005759; WO2007046729; WO2008145116; WO2015063553; WO2017106803; and WO2018162581, which are herein incorporated by reference.

As used herein, “Mito-MES” is used to refer to mitoquinone (10-(4,5-dimethoxy-2-methyl-3,6-dioxo-1,4-cyclohexadienyl) decyl triphenylphosphonium) mesylate and/or mitoquinol (10-(4,5-dimethoxy-2-methyl-3,6-dihydroxy-1,4-cyclohexadienyl) decyl triphenylphosphonium) mesylate, and the term “mitoquin” is used to refer to mitoquinone and/or mitoquinol. It should be noted that in vivo mitoquinone is rapidly distributed to tissues and then converted into mitoquinol within cells and either mitoquinone or mitoquinol may be used by cells in vitro. For convenience, the term “Mito-MES” is used throughout the experiments described herein; however, mitoquinone mesylate was used for the in vitro experiments and mitoquinol mesylate was used for the in vivo animal studies, unless specifically indicated otherwise.

Derivatives of mitoquinone and mitoquinol refer to compounds having the following structural formula as part of its backbone structure:

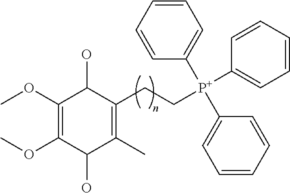

wherein “n” is any number, preferably n is 1-15, more preferably n is 5-10, most preferably n is 9.

As used herein, a “3CLpro inhibitor” is used to refer to inhibitors of 3C-like protease inhibitors. Exemplary 3CLpro inhibitors include nirmatrelvir compounds and the inhibitors described in He et al. (2020) Int J Antimicrob Agents 56(2):106055, which is herein incorporated by reference in its entirety. As used herein, a “nirmatrelvir compound” refers to nirmatrelvir and derivatives thereof such as those described in U.S. Ser. No. 11/358,953, which is herein incorporated by reference in its entirety.

As used herein, a “protease inhibitor” refers to an agent that inhibits the activity of a protease. Exemplary protease inhibitors include ritonavir compounds, atazanavir (REYATAz), darunavir (PREzisTA), fosamprenavir (LExivA), indinavir (CRixivAN), lopinavir/ritonavir (KALETRA), nelfinavir (VIRACEPT), saquinavir (INviRAsE), tipranavir (APTIvus), atazanavir/cobicistat (EvoTAz), darunavir/cobicistat (PREzcomx), and derivatives thereof. As used herein, a “ritonavir compound” refers to ritonavir (NoRviR) and derivatives thereof such as that described in U.S. Pat. Nos. 5,354,866, 5,541,206, the analogous compounds described in U.S. Pat. No. 7,763,733, which are herein incorporated by reference in their entirety.

Exemplary “N⁴-hydroxycytidine nucleosides” include molnupiravir and the nucleosides and derivatives described in U.S. Pat. No. 9,809,616, U.S. Ser. No. 10/874,683, U.S. Ser. No. 11/147,826, U.S. Ser. No. 11/197,882, U.S. Ser. No. 11/312,743, U.S. Ser. No. 11/331,331, US20200276219, and US20220185840, which are herein incorporated by reference in their entirety.

As used herein, the terms “subject”, “patient”, and “individual” are used interchangeably to refer to humans and non-human animals. The terms “non-human animal” and “animal” refer to all non-human vertebrates, e.g., non-human mammals and non-mammals, such as non-human primates, horses, sheep, dogs, cows, pigs, chickens, and other veterinary subjects and test animals. In some embodiments, the subject is a mammal. In some embodiments, the subject is a human.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

As used herein, the phrase “consists essentially of” in the context of a given ingredient in a composition, means that the composition may include additional ingredients so long as the additional ingredients do not adversely impact the activity, e.g., biological or pharmaceutical function, of the given ingredient.

The phrase “comprises, consists essentially of, or consists of A” is used as a tool to avoid excess page and translation fees and means that in some embodiments the given thing at issue: comprises A, consists essentially of A, or consists of A. For example, the sentence “In some embodiments, the composition comprises, consists essentially of, or consists of A” is to be interpreted as if written as the following three separate sentences: “In some embodiments, the composition comprises A. In some embodiments, the composition consists essentially of A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

### Additional Embodiments

Embodiment 1: A method of treating, inhibiting, and/or reducing an infection by a virus or a symptom caused by the infection in a subject, which comprises, consists essentially of, or consists of administering (a) one or more Nrf2 agonists; and (b) one or more 3CLpro inhibitors, one or more protease inhibitors, and/or one or more N⁴-hydroxycytidine nucleosides to the subject.

Embodiment 2: The method according to Embodiment 1, wherein the one or more Nrf2 agonists is a TPP Compound, a mitochondrial targeted antioxidant, or dimethyl fumarate (DMF).

Embodiment 3: The method according to Embodiment 1, wherein the one or more Nrf2 agonists is mitoquinone mesylate and/or mitoquinol mesylate.

Embodiment 4: The method according to Embodiment 1, wherein the one or more Nrf2 agonists is DMF.

Embodiment 5: The method according to Embodiment 1, wherein the one or more Nrf2 agonists comprises, consists essentially of, or consists of (i) DMF, and (ii) mitoquinone mesylate and/or mitoquinol mesylate.

Embodiment 6: The method according to any one of Embodiments 1 to 5, wherein the 3CLpro inhibitor is a nirmatrelvir compound.

Embodiment 7: The method according to any one of Embodiments 1 to 6, wherein the protease inhibitor is a ritonavir compound.

Embodiment 8: The method according to any one of Embodiments 1 to 7, wherein N⁴-hydroxycytidine nucleoside is molnupiravir.

Embodiment 9: The method according to any one of Embodiments 1 to 8, wherein the virus is a coronavirus.

Embodiment 10: The method according to any one of Embodiments 1 to 9, wherein the symptom caused by the infection is cytotoxic injury, an aberrant host inflammatory response, and/or lung injury.

Embodiment 11: A composition, which comprises, consists essentially of, or consists of (a) one or more Nrf2 agonists; and (b) one or more 3CLpro inhibitors, one or more protease inhibitors, and/or one or more N⁴-hydroxycytidine nucleosides.

Embodiment 12: The composition according to Embodiment 11, wherein the one or more Nrf2 agonists is a TPP Compound, a mitochondrial targeted antioxidant, or dimethyl fumarate (DMF).

Embodiment 13: The composition according to Embodiment 11, wherein the one or more Nrf2 agonists is mitoquinone mesylate and/or mitoquinol mesylate.

Embodiment 14: The composition according to Embodiment 11, wherein the one or more Nrf2 agonists is DMF.

Embodiment 15: The composition according to Embodiment 11, wherein the one or more Nrf2 agonists comprises, consists essentially of, or consists of (i) DMF, and (ii) mitoquinone mesylate and/or mitoquinol mesylate.

Embodiment 16: The composition according to any one of Embodiments 11 to 15, wherein the 3CLpro inhibitor is a nirmatrelvir compound.

Embodiment 17: The composition according to any one of Embodiments 11 to 16, wherein the protease inhibitor is a ritonavir compound.

Embodiment 18: The composition according to any one of Embodiments 11 to 17, wherein N⁴-hydroxycytidine nucleoside is molnupiravir.

Embodiment 19: A kit, which comprises, consists essentially of, or consists of (a) one or more Nrf2 agonists; and (b) one or more 3CLpro inhibitors, one or more protease inhibitors, and/or one or more N⁴-hydroxycytidine nucleosides packaged together.

Embodiment 20: The kit according to Embodiment 19, wherein the one or more Nrf2 agonists is a TPP Compound, a mitochondrial targeted antioxidant, or dimethyl fumarate (DMF).

Embodiment 21: The kit according to Embodiment 19, wherein the one or more Nrf2 agonists is mitoquinone mesylate and/or mitoquinol mesylate.

Embodiment 22: The kit according to Embodiment 19, wherein the one or more Nrf2 agonists is DMF.

Embodiment 23: The kit according to Embodiment 19, wherein the one or more Nrf2 agonists comprises, consists essentially of, or consists of (i) DMF, and (ii) mitoquinone mesylate and/or mitoquinol mesylate.

Embodiment 24: The kit according to any one of Embodiments 19 to 23, wherein the 3CLpro inhibitor is a nirmatrelvir compound.

Embodiment 25: The kit according to any one of Embodiments 19 to 24, wherein the protease inhibitor is a ritonavir compound.

Embodiment 26: The kit according to any one of Embodiments 19 to 25, wherein N⁴-hydroxycytidine nucleoside is molnupiravir.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

What is claimed is:

1. A composition, which comprises, consists essentially of, or consists of (a) one or more Nrf2 agonists; and (b) one or more 3CLpro inhibitors, one or more protease inhibitors, and/or one or more N⁴-hydroxycytidine nucleosides.

2. The composition according to claim 1, wherein the one or more Nrf2 agonists is
a) a TPP Compound, a mitochondrial targeted antioxidant, or dimethyl fumarate (DMF);
b) mitoquinone mesylate and/or mitoquinol mesylate;
c) DMF; or
d) (i) DMF, and (ii) mitoquinone mesylate and/or mitoquinol mesylate.

3. The composition according to claim 1, wherein
the 3CLpro inhibitor is a nirmatrelvir compound,
the protease inhibitor is a ritonavir compound, and/or
the N⁴-hydroxycytidine nucleoside is molnupiravir.

4. The composition according to claim 2, wherein
the 3CLpro inhibitor is a nirmatrelvir compound,
the protease inhibitor is a ritonavir compound, and/or
the N⁴-hydroxycytidine nucleoside is molnupiravir.

5. The composition according to claim 1, wherein said composition comprises (a) a TPP Compound, (b) DMF, and (c)(i) nirmatrelvir and ritonavir, or (ii) molnupiravir.

6. A method of treating, inhibiting, and/or reducing an infection by a virus or a symptom caused by the infection in a subject, which comprises, consists essentially of, or consists of administering (a) one or more Nrf2 agonists; and (b) one or more 3CLpro inhibitors, one or more protease inhibitors, and/or one or more N⁴-hydroxycytidine nucleosides to the subject.

7. The method according to claim 6, wherein the one or more Nrf2 agonists is
a) a TPP Compound, a mitochondrial targeted antioxidant, or dimethyl fumarate (DMF);
b) mitoquinone mesylate and/or mitoquinol mesylate;
c) DMF; or
d) (i) DMF, and (ii) mitoquinone mesylate and/or mitoquinol mesylate.

8. The method according to claim 6, wherein
the 3CLpro inhibitor is a nirmatrelvir compound,
the protease inhibitor is a ritonavir compound, and/or
the N⁴-hydroxycytidine nucleoside is molnupiravir.

9. The method according to claim 7, wherein
the 3CLpro inhibitor is a nirmatrelvir compound,
the protease inhibitor is a ritonavir compound, and/or
the N⁴-hydroxycytidine nucleoside is molnupiravir.

10. The method according to claim 6, wherein said composition comprises (a) a TPP Compound, (b) DMF, and (c)(i) nirmatrelvir and ritonavir, or (ii) molnupiravir.

11. The method according to claim 6, wherein the virus is a coronavirus.

12. The method according to claim 6, wherein the symptom caused by the infection is cytotoxic injury, an aberrant host inflammatory response, and/or lung injury.

13. A kit, which comprises, consists essentially of, or consists of (a) one or more Nrf2 agonists; and (b) one or more 3CLpro inhibitors, one or more protease inhibitors, and/or one or more N⁴-hydroxycytidine nucleosides packaged together.

14. The kit according to claim 13, wherein the one or more Nrf2 agonists is
a) a TPP Compound, a mitochondrial targeted antioxidant, or dimethyl fumarate (DMF);
b) mitoquinone mesylate and/or mitoquinol mesylate;
c) DMF; or
d) (i) DMF, and (ii) mitoquinone mesylate and/or mitoquinol mesylate.

15. The kit according to claim 13, wherein
the 3CLpro inhibitor is a nirmatrelvir compound,
the protease inhibitor is a ritonavir compound, and/or
the N⁴-hydroxycytidine nucleoside is molnupiravir.

16. The kit according to claim 14, wherein
the 3CLpro inhibitor is a nirmatrelvir compound,
the protease inhibitor is a ritonavir compound, and/or
the N⁴-hydroxycytidine nucleoside is molnupiravir.

17. The kit according to claim 13, wherein the kit comprises, packaged together, (a) a TPP Compound, (b) DMF, and (c)(i) nirmatrelvir and ritonavir, or (ii) molnupiravir.
